(19) United States
(12) Patent Application Publication
(10) Pub. No.: US 2014/0357654 A1
(43) Pub. Date: Dec. 4, 2014

(54) METHODS OF TREATING SCHIZOPHRENIA WITH PHENYLALANINE ENAMIDE DERIVATIVE INHIBITORS OF a4 INTEGRIN POSSESSING A CYCLOBUTENE GROUP

(71) Applicant: AESTUS THERAPEUTICS, INC., East Windsor, NJ (US)

(72) Inventors: Tage Honore, Princeton, NJ (US); Meredith H. Prysak, Monroe Township, NJ (US)

(73) Assignee: AESTUS THERAPEUTICS, INC., East Windsor, NJ (US)

(21) Appl. No.: 14/356,398

(22) PCT Filed: Nov. 8, 2012
(86) PCT No.: PCT/US2012/064054

§ 371 Date: May 6, 2014

Related U.S. Application Data
(60) Provisional application No. 61/557,631, filed on Nov. 9, 2011.

(51) Int. Cl. C07D 471/04

(52) U.S. Cl. CPC C07D 471/04
USPC 514/278

(57) ABSTRACT

Embodiments of the invention relate to the treatment of schizophrenia in mammals. Embodiments of the invention include methods for treating schizophrenia and/or symptoms of schizophrenia and/or a positive symptom of schizophrenia in a psychotic disease as well as methods for preparing medicaments used in the treatment of mammalian schizophrenia. In one embodiment, methods of the invention comprise the inhibition of alpha4 integrin by a genus of compounds for the treatment of mammalian schizophrenia or a positive symptom of schizophrenia in a psychotic disease.

### FIELD

Embodiments of the invention relate to the treatment of schizophrenia in mammals.

### BACKGROUND

### Schizophrenia

Schizophrenia is a mental disorder marked by severe cognitive dysfunction and psychosis, estimated to affect greater than 1% of the population, with onset typically in late adolescence and early adulthood. There are three main symptom categories of schizophrenia: positive symptoms, which include delusions, hallucinations, and movement and thought disorders, such as disorganized speech and/or grossly disorganized or catatonic behavior; negative symptoms comprised of social disturbances, flat affect and deficits in expressing emotion and experiencing pleasure such as alogia and/or avolition; and cognitive symptoms, which affect attention span, working memory and other intellectual functions and may include one or more of disorganized thinking, slow thinking, difficulty understanding, poor concentration, poor memory, difficulty expressing thoughts and difficulty integrating thoughts, feelings and behavior. Together, these symptoms have a severe impact on the quality of life for the schizophrenic patient, and contribute to a higher rate of substance abuse and suicide, factors contributing to a life expectancy of fifteen years less than the norm.

The positive symptoms of schizophrenia may also be present in other psychotic diseases, for example, bipolar disorder, delusional disorder, psychotic depression, Tourette syndrome, autism spectrum disorder, OCD, dementia and Alzheimer's disease. Antipsychotics used to treat these symptoms of schizophrenia may also used to treat these symptoms in the other pshychotic diseases.

Schizophrenia is a clear unmet medical need, of great import to general public health. Finding effective treatments for this disease has been difficult because both the disease course and cause are highly complex, comprising any number of poorly understood genetic and environmental interactions. Currently, schizophrenia is managed using second-generation, or “atypical”, anti-psychotic agents. These dopamine receptor antagonists target serotonin receptors to a greater extent than their predecessors, and may target other receptors such as histamine and NMDA receptors. They are more effective at treating positive symptoms, but are not effective against negative or cognitive deficit symptoms. Thus, there is an urgent need for new classes of schizophrenia drugs.

### CDP-323 and Related α4 Integrin Inhibitors

UCB Pharma S.A. and Biogen Idec collaborated from 2007 to 2010 to perform human clinical testing of an oral formulation of the small-molecule CDP-323 (also known as Zaurategrast) for the potential treatment of multiple sclerosis (see clinical trials NCT00484536 and NCT00726648 listed by U.S. National Institutes of Health at www.clinicaltrials.gov, as displayed on Jul. 18, 2011). CDP-323 is (2S)-2-(2-Bromo-3-oxo-spiro[3.5]non-1-en-1-ylamino)-3-[4-([2,7]naphthyridin-1-ylamino)phenyl]proprionic acid and has the following structure:

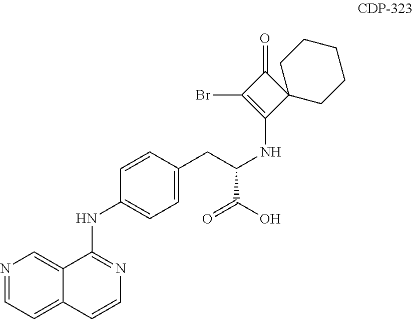 [SMILES: O=C(O)[C@H](Cc1ccc(Nc2nccc3ccncc23)cc1)NC1=C(Br)C(=O)C12CCCCC2]

U.S. Pat. No. 7,501,437 discloses a genus of α4 integrin inhibitors, including (2S)-2-(2-Bromo-3-oxo-spiro[3.5]non-1-en-1-ylamino)-3-[4-([2,7]naphthyridin-1-ylamino)phenyl]proprionic acid, that are useful for the treatment of diseases or disorders, including inflammation, in which the extravasation of leukocytes plays a role, such as multiple sclerosis, allograft rejection, asthma and inflammatory bowel disease. U.S. Pat. No. 6,835,738 discloses a genus of esters of (2S)-2-(2-Bromo-3-oxo-spiro[3.5]non-1-en-1-ylamino)-3-[4-([2,7]naphthyridin-1-ylamino)phenyl]proprionic acid, which are also useful in the treatment of diseases or disorders, including inflammation, in which the extravasation of leukocytes plays a role, such as multiple sclerosis, allograft rejection, asthma and inflammatory bowel disease. U.S. Pat. Nos. 7,501,437 and 6,835,738 teach how to manufacture, pharmaceutically formulate and administer these α4 integrin inhibitors, including (2S)-2-(2-Bromo-3-oxo-spiro[3.5]non-1-en-1-ylamino)-3-[4-([2,7]naphthyridin-1-ylamino)phenyl]proprionic acid, for the treatment of diseases or disorders, including inflammation, in which the extravasation of leukocytes plays a role, such as multiple sclerosis, allograft rejection, asthma and inflammatory bowel disease. U.S. Pat. Nos. 7,501,437 and 6,835,738 are hereby incorporated herein in their entirety.

In particular, U.S. Pat. No. 7,501,437 discloses a genus of α4 integrin inhibitors of Formula 1, as reproduced below:

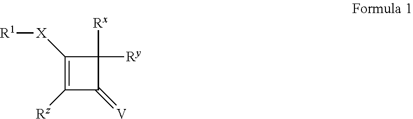

wherein
R¹ is a group Ar¹L²Ar²Alk- in which:
   Ar¹ is an optionally substituted aromatic or heteroaromatic group;
   L² is a covalent bond or a linker atom or group;
   Ar² is an optionally substituted arylene or heteroarylene group; and
   Alk is a chain —CH₂CH(R)—, —CH═C(R)—, or

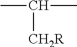

in which R is a carboxylic acid (—CO₂H) or a derivative or biostere thereof;
X is an —O— or —S— atom or N(R²) group in which:
   R² is a hydrogen atom or a C₁₋₆alkyl group;
V is an oxygen (O) or sulfur (S) atom;
R^{x}, R^{y} and R^{z}, which may be the same or different, is each an atom or group -L¹(Alk¹)ₙ(R³)ᵥ in which L¹ is a covalent bond or a linker atom or group, Alk¹ is an optionally substituted aliphatic or heteroaliphatic chain, R³ is a hydrogen or halogen atom or group selected from —OR^{3a} [where R^{3a} is a hydrogen atom or an optionally substituted straight or branched C₁₋₆alkyl group or C₃₋₈cycloalkyl group], SR^{3a}, CN or an optionally substituted cycloaliphatic, heterocycloaliphatic, polycycloaliphatic, heteropolycycloaliphatic, aromatic or heteroaromatic group, n is zero or the integer 1 and v is the integer 1, 2 or 3 provided that when n is zero and L¹ is a covalent bond v is the integer 1;
or Rz is an atom or group as previously defined and R^{x} and R^{y} are joined together to form an optionally substituted spiro linked cycloaliphatic or heterocycloaliphatic group.

U.S. Pat. No. 7,501,437 discloses another genus of α4 integrin inhibitors of Formula 1, wherein:
R¹ is a group Ar¹L²Ar²Alk-;
Ar¹ is a naphthyridynyl group optionally substituted with one or more -L³(Alk²)ₜL⁴(R⁴)ᵤ atoms or groups;
L³ and L⁴ are each, independently, a covalent bond, —O—, —S—, —C(O)—, —C(O)O—, —OC(O)—, —C(S)—, —S(O)—, —S(O)₂—, —N(R⁸)—, —CON(R⁸)—, —OC(O)N(R⁸)—, —CSN(R⁸)—, —N(R⁸)CO—, —N(R⁸)C(O)O—, —N(R⁸)CS—, —S(O)₂N(R⁸)—, —N(R⁸)S(O)₂—, —N(R⁸)O—, —ON(R⁸)—, —N(R⁸)N(R⁸)—, —N(R⁸)CON(R⁸)—, —N(R⁸)CSN(R⁸)—, or —N(R⁸)SO₂N(R⁸)—;
R⁸ is a hydrogen atom or a straight or branched C₁₋₆alkyl group optionally substituted with one, two, or three substituents selected from halogen, hydroxy and C₁₋₆alkoxy;
t is zero or the integer 1;
u is an integer 1, 2 or 3;
Alk² is an aliphatic or heteroaliphatic chain optionally substituted with one or more substituents selected from halogen, —OH, —CO₂H, —CO₂R⁹, —CONHR⁹, —CON(R⁹)₂, —COR⁹, C₁₋₆alkoxy, thiol, —S(O)R⁹, —S(O)₂R⁹, C₁₋₆ alkylthio, amino, —NHR⁹ and —N(R⁹)₂;
R⁹ is a straight or branched C₁₋₆alkyl group optionally substituted with one, two, or three substituents selected from halogen, hydroxy and C₁₋₆alkoxy;
R⁴ is a hydrogen atom; a halogen atom; C₁₋₆alkyl optionally substituted with one, two, or three substituents selected from halogen, hydroxy and C₁₋₆alkoxy; C₃₋₈cycloalkyl optionally substituted with one, two, or three substituents selected from halogen, hydroxy and C₁₋₆alkoxy; —OR⁵; —SR⁵; —NR⁵R⁶; —NO₂; —CN; —CO₂R⁵; —SO₃H; —SOR⁵; —SO₂R⁵; —SO₃R⁵; —OCO₂R⁵; —CONR⁵R⁶; —OCONR⁵R⁶; —CSNR⁵R⁶; —COR⁵; —OCOR⁵; —N(R⁵)COR⁶; —N(R⁵)CSR⁶; —SO₂N(R⁵)(R⁶); —N(R⁵)SO₂R⁶; N(R⁵)CON(R⁶)(R⁷); —N(R⁵)CSN(R⁶)(R⁷); or —N(R⁵)SO₂N(R⁶)(R⁷), provided that when t is zero and each of L³ and L⁴ is a covalent bond, then u is the integer 1 and R⁴ is other than a hydrogen atom;
R⁵, R⁶, R⁷, and R¹¹ are each, independently, a hydrogen atom or a C₁₋₆alkyl or C₃₋₈cycloalkyl group, wherein each of said alkyl and cycloalkyl groups is optionally substituted with one, two, or three substituents selected from halogen, hydroxy and C₁₋₆alkoxy;
L² is an N(R⁸) group;
Ar² is an arylene or heteroarylene group optionally substituted with one or more -L³(Alk²)ₜL⁴(R⁴)ᵤ atoms or groups;
Alk is a chain —CH₂—CH(R)—, —CH═C(R)—, or

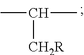

R is a carboxylic acid (—CO₂H), a carboxylic acid ester (—CO₂Alk⁷), a carboxylic acid amide (—CONR⁵R⁶), a tetrazole, phosphonic acid, phosphinic acid, sulphonic acid, sulphinic acid, boronic acid, or an acylsulphonamide group;
Alk⁷ is a straight or branched C₁₋₈alkyl group, C₂₋₈alkenyl group, C₂₋₈alkynyl group, C₃₋₈cycloalkyl group, C₃₋₈heterocycloalkyl group, C₃₋₈cycloalkylC₁₋₈alkyl group, C₃₋₈heterocycloalkylC₁₋₈alkyl group, C₁₋₆alkyloxyC₁₋₆alkyl group, hydroxyC₁₋₆alkyl group, C₁₋₆alkylthioC₁₋₆alkyl group, C₁₋₆alkylsulfinylC₁₋₆alkyl group, C₁₋₆alkylsulfonylC₁₋₆alkyl group, C₃₋₈cycloalkyloxyC₁₋₆alkyl group, C₃₋₈cycloalkylthioC₁₋₆alkyl group, C₃₋₈cycloalkylsulfinylC₁₋₆alkyl group, C₃₋₈cycloalkylsulfonylC₁₋₆alkyl group, C₁₋₆alkyloxycarbonylC₁₋₆alkyl group, C₁₋₆alkyloxycarbonylC₁₋₆alkenyl group, C₁₋₆alkyloxycarbonyloxyC₁₋₆alkyl group, C₁₋₆alkyloxycarbonyloxyC₁₋₆alkenyl group, C₃₋₈cycloalkyloxycarbonyloxyC₁₋₆alkyl group, N-di-C₁₋₈alkylaminoC₁₋₈alkyl group, N—C₆₋₁₂aryl-N—C₁₋₆alkylaminoC₁₋₆alkyl group, N-di-C₁₋₈alkyl-carbamoylC₁₋₈alkyl group, C₆₋₁₂arylC₁₋₆alkyl group, heteroC₆₋₁₀arylC₁₋₆alkyl group, C₆₋₁₂aryl group, a C₆₋₁₂aryloxyC₁₋₈alkyl group, a C₆₋₁₂arylthioC₁₋₈alkyl group, a C₆₋₁₂arylsulfinylC₁₋₈alkyl group, a C₆₋₁₂arylsulfonylC₁₋₈alkyl group, C₁₋₈alkanoyloxyC₁₋₈alkyl group, C₄₋₈imidoC₁₋₈alkyl group, a C₆₋₁₂aroyloxyC₁₋₈alkyl group, or a triglyceride, optionally substituted with one or more R^{13a} groups;
R^{13a} is a halogen atom, or an amino (—NH₂), NHR¹⁴, —N(R¹⁴)₂, nitro, cyano, amidino, hydroxyl (—OH), —OR¹⁴, formyl, carboxyl (—CO₂H), esterified carboxyl, thiol (—SH), —SR¹⁴, SC(═NH)NH₂, —COR¹⁴, CSR¹⁴, —SO₃H, —SOR¹⁴, —SO₂R¹⁴, —SO₃R¹⁴, —SO₂NH₂, —SO₂NHR¹⁴, —SO₂N(R¹⁴)₂, —CONH₂, —CSNH₂, —CONHR¹⁴, —CSNHR¹⁴, —CON(R¹⁴)₂, —CSN(R¹⁴)₂, —N(R¹¹)SO₂R¹⁴, —N(SO₂R¹⁴)₂, —NH(R¹¹)SO₂NH₂, —N(R¹¹)SO₂NHR¹⁴, —N(R¹¹)SO₂N(R¹⁴)₂, —N(R¹¹)COR¹⁴, —N(R¹¹)CONH₂, —N(R¹¹)CONHR¹⁴, —N(R¹¹)CON(R¹⁴)₂, N(R¹¹)CSNH₂, —N(R¹¹)CSNHR114, N(R¹¹)CSN(R¹⁴)₂, —N(R¹¹)CSR¹⁴, —N(R¹¹)C(O)OR¹⁴, —SO₂NHet¹, —CONHet¹, —CSNHet¹, —N(R¹¹)SO₂NHet¹, —N(R¹¹)CONHet¹, —N(R¹¹)CSNHet¹, —SO₂N(R¹¹)Het², -Het², —CON(R¹¹)Het², —CSN(R¹¹)Het², —N(R¹¹)CON(R¹¹)Het², —N(R¹¹)CSN Het², aryl or heteroaryl group;
R¹⁴ is an -Alk⁶(R^{13a})ₘ, aryl, or heteroaryl group;
Alk⁶ is a straight or branched C₁₋₆alkylene, C₂₋₆alkenylene or C₂₋₆alkynylene chain, optionally interrupted by one, two or three —O— or —S— atoms or —S(O)ₚ or —N(R¹⁵)— groups;
m is zero or the integer 1, 2 or 3;
p is an integer 1 or 2;
R¹⁵ is a hydrogen atom or C₁₋₆alkyl group;
Het¹ is a C₅₋₇cyclicamino group optionally containing one or more —O— or —S— atoms or —N(R¹¹)—, —C(O)—, —C(S)—, —S(O) or —S(O)₂ groups and optionally substituted with one or more substituents selected from halogen, —OH, —CO₂H, —CO₂R⁹, CONHR⁹, —CON(R⁹)₂, —COR⁹, C₁₋₆alkoxy, thiol, —S(O)R⁹, —S(O)₂R⁹, C₁₋₆alkylthio, amino, —NHR⁹ and —N(R⁹)₂;
Het² is a monocyclic C₅₋₇carbocyclic group optionally containing one or more —O— or —S— atoms or —N(R¹¹)—, —C(O)— or —C(S)— groups and optionally substituted with one or more substituents selected from halogen, —OH, —CO₂H, —CO₂R⁹, —CONHR⁹, —CON(R⁹)₂, —COR⁹, C₁₋₆alkoxy, thiol, —S(O)R⁹, —S(O)₂R⁹, C₁₋₆alkylthio, amino, —NHR⁹ and —N(R⁹)₂;
X is an —O— or —S— atom or an —N(R²)— group;
R² is a hydrogen atom or a C₁₋₆alkyl group;
V is an oxygen (O) or sulfur (S) atom;
R^{z} is an atom or group -L¹(Alk¹)ₙ(R³)ᵥ;
L¹ is a covalent bond, an —O—, —S—, or —Se— atom, or a —C(O)—, —C(O)O—, —OC(O)—, —C(S)—, —S(O)—, —S(O)₂—, —N(R⁸)—, —CON(R⁸)—, —OC(O)N(R⁸)—, —CSN(R⁸)—, —N(R⁸)CO—, —N(R⁸)C(O)O—, —N(R⁸)CS—, S(O)₂N(R⁸)—, —N(R⁸)S(O)₂—, —N(R⁸)O—, —ON(R⁸)—, —N(R⁸)N(R⁸)—, —N(R⁸)CON(R⁸)—, —N(R⁸)CSN(R⁸)—, or —N(R⁸)SO₂N(R⁸)— group;
Alk¹ is an aliphatic or heteroaliphatic chain optionally substituted with one or more substituents selected from halogen, —OH, —CO₂H, —CO₂R⁹, —CONHR⁹, —CON(R⁹)₂, —COR⁹, C₁₋₆alkoxy, thiol, —S(O)R⁹, —S(O)₂R⁹, C₁₋₆alkylthio amino, —NHR⁹ and —N(R⁹)₂;
R³ is a hydrogen or halogen atom or a group selected from —OR^{3a}, —SR^{3a}, —CN and a C₃₋₁₀cycloalkyl; C₃₋₁₀cycloalkenyl; C₃₋₁₀heterocycloalkyl or C₃₋₁₀heterocycloalkenyl containing 1, 2, 3 or 4 heteroatoms or heteroatom-containing groups L⁵, where L⁵ is defined as for L³; aromatic or heteroaromatic group optionally substituted with one or more substituents selected from halogen, C₁₋₆alkyl, haloC₁₋₆alkyl, —C(OH)(CF₃)₂, C₁₋₆alkoxy, haloC₁₋₆alkoxy, thiol, C₁₋₆alkylthio, -(Alk⁴)_{g}R¹⁰, —CN, —CO₂R¹¹, —NO₂, —CON(R¹¹)₂, —CSN(R¹¹)₂, —COR¹¹, —CSN(R¹¹)₂, —N(R¹¹)COR¹¹, —N(R¹¹)CSR¹¹, —SO₂N(R¹¹)₂, N(R¹¹)SO₂R¹¹, —N(R¹¹)CON(R¹¹)₂, —N(R¹¹)CSN(R¹¹), N(R¹¹)SO₂N(R¹¹)₂, and phenyl optionally substituted with one, two or three R¹³ groups;
R¹³ is —R^{13a} or -Alk⁶(R^{13a})ₘ;
Alk⁴ is a straight or branched C₁₋₃alkylene chain;
g is zero or an integer 1;
R¹⁰ is —OH, —SH, or —N(R¹¹)₂;
R^{3a} is a hydrogen atom or a straight or branched C₁₋₆alkyl group or C₃₋₈cycloalkyl group, wherein each of said alkyl and cycloalkyl groups is optionally substituted with one, two, or three substituents selected from halogen, hydroxy, and C₁₋₆alkoxy;
n is zero or the integer 1;
v is the integer 1, 2 or 3;
provided that when n is zero and L¹ is a covalent bond, v is the integer 1;
R^{X} and R^{Y}, together with the carbon atom to which they are attached, are joined together to form a spiro linked cyclopentyl, cyclohexyl, cycloheptyl, or tetrahydropyranyl group optionally substituted with one or more substituents selected from halogen, C₁₋₆alkyl, haloC₁₋₆alkyl, —C(OH)(CF₃)₂, C₁₋₆alkoxy, haloC₁₋₆alkoxy, thiol, C₁₋₆alkylthio, -(Alk⁴)_{g}R¹⁰, —CN, —CO₂R¹¹, —NO₂, —CON(R¹¹)₂, —CSN(R¹¹)₂, —COR¹¹, —CSN(R¹¹)₂, —N(R¹¹)COR¹¹, —N(R¹¹)CSR¹¹, —SO₂N(R¹¹)₂, —N(R¹¹)SO₂R¹¹, —N(R¹¹)CON(R¹¹)₂, —N(R¹¹)CSN(R¹¹), N(R¹¹)SO₂N(R¹¹)₂, and phenyl optionally substituted with one, two or three R¹³ groups.

U.S. Pat. Nos. 7,501,437; 6,835,738 and 6,878,718 disclose a genus of α4 integrin inhibitors of Formula 2:

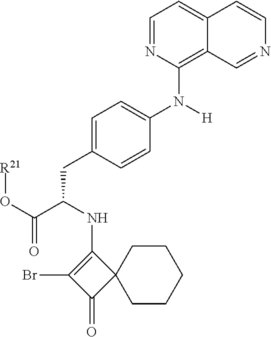

wherein R²¹ is H; a straight or branched C₁₋₆alkyl group optionally substituted with one, two or three substituents selected from halogen, hydroxy and C₁₋₆alkoxy; —CH₂CH₂OCH₃; —CH₂CH₂OCH₂CH₂OH; —CH₂CH₂OCH₂CH₂OCH₃;

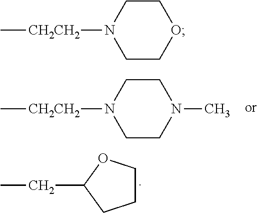

It will be appreciated that compounds of Formula 1 and Formula 2 may have one or more chiral centers, and exist as enantiomers or diastereomers. The invention is to be understood to extend to all such enantiomers, diastereomers and mixtures thereof, including racemates. Formula 1 and Formula 2 are intended to represent all individual isomers and mixtures thereof, unless stated or shown otherwise. In addition, compounds of Formula 1 and Formula 2 may exist as tautomers, for example keto (CH₂C═O)-enol (CH═CHOH) tautomers. Formula 1 and Formula 2 are intended to represent all individual tautomers and mixtures thereof unless stated otherwise.

Optionally substituted aromatic groups represented by Ar¹ when present in the group R¹ include for example optionally substituted monocyclic or bicyclic fused ring C₆₋₁₂aromatic groups, such as phenyl, 1- or 2-naphthyl, 1- or 2-tetrahydronaphthyl, indanyl or indenyl groups.

Optionally substituted heteroaromatic groups represented by the group Ar¹ when present in the group R¹ include for example optionally substituted C₁₋₉heteroaromatic groups containing for example one, two, three or four heteroatoms selected from oxygen, sulfur or nitrogen atoms. In general, the heteroaromatic groups may be for example monocyclic or bicyclic fused ring heteroaromatic groups. Monocyclic heteroaromatic groups include for example five- or six-membered heteroaromatic groups containing one, two, three or four heteroatoms selected from oxygen, sulfur or nitrogen atoms. Bicyclic heteroaromatic groups include for example eight- to thirteen-membered fused-ring heteroaromatic groups containing one, two or more heteroatoms selected from oxygen, sulfur or nitrogen atoms.

Particular examples of heteroaromatic groups of these types include pyrrolyl, furyl, thienyl, imidazolyl, N—C₁₋₆alkylimidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-thiadiazole, pyridyl, pyrimidinyl, pyridazinyl, pyrazinyl, 1,3,5-triazinyl, 1,2,4-triazinyl, 1,2,3-triazinyl, benzofuryl, [2,3-dihydro]benzofuryl, [2,3-dihydro]benzothienyl, benzothienyl, benzotriazolyl, indolyl, isoindolyl, benzimidazolyl, imidazo[1,2-a]pyridyl, benzothiazolyl, benzoxazolyl, benzisoxazolyl, benzopyranyl, [3,4-dihydro]benzopyranyl, quinazolinyl, quinoxalinyl, naphthyridinyl, e.g. 2,6-naphthyridinyl, or 2,7-naphthyridinyl, pyrido[3,4-b]pyridyl, pyrido[3,2-b]pyridyl, pyrido[4,3-b]pyridyl, quinolinyl, isoquinolinyl, tetrazolyl, 5,6,7,8-tetrahydroquinolinyl, 5,6,7,8-tetrahydroisoquinolinyl, and imidyl, e.g. succinimidyl, phthalimidyl, or naphthalimidyl such as 1,8-naphthalimidyl.

Each aromatic or heteroaromatic group represented by the group Ar¹ may be optionally substituted on any available carbon or, when present, nitrogen atom. One, two, three or more of the same or different substituents may be present and each substituent may be selected for example from an atom or group -L³(Alk²)ₜL⁴(R⁴)ᵤ in which L³ and L⁴, which may be the same or different, is each a covalent bond or a linker atom or group, t is zero or the integer 1, u is an integer 1, 2 or 3, Alk² is an optionally substituted aliphatic or heteroaliphatic chain and R⁴ is a hydrogen or halogen atom or a group selected from optionally substituted C₁₋₆alkyl or C₃₋₈cycloalkyl, —OR⁵ [where R⁵ is a hydrogen atom, an optionally substituted C₁₋₆alkyl or C₃₋₈cycloalkyl group], —SR⁵, —NR⁵R⁶ [where R⁶ is as just defined for R⁵ and may be the same or different], —NO₂, —CN, —CO₂R⁵, SO₃H, —SOR⁵, —SO₂R⁵, —SO₃R⁵, —OCO₂R⁵, —CONR⁵R⁶, —OCONR⁵R⁶, —CSNR⁵R⁶, —COR⁵, —OCOR⁵, —N(R⁵)COR⁶, —N(R⁵)CSR⁶, —SO₂N(R⁵)(R⁶), —N(R⁵)SO₂R⁶, N(R⁵)CON(R⁶)(R⁷) [where R⁷ is a hydrogen atom, an optionally substituted C₁₋₆alkyl or C₃₋₈cycloalkyl group], —N(R⁵)CSN(R⁶)(R⁷) or —N(R⁵)SO₂N(R⁶)(R⁷), provided that when t is zero and each of L³ and L⁴ is a covalent bond then u is the integer 1 and R⁴ is other than a hydrogen atom.

When L³ and/or L⁴ is present in these substituents as a linker atom or group it may be any divalent linking atom or group. Particular examples include —O— or —S— atoms or —C(O), —C(O)O—, —OC(O)—, —C(S)—, —S(O)—, —S(O)₂—, —N(R⁸)— [where R⁸ is a hydrogen atom or an optionally substituted straight or branched C₁₋₆alkyl group], —CON(R⁸)—, —OC(O)N(R⁸)—, —CSN(R⁸)—, —N(R⁸)CO—, —N(R⁸)C(O)O—, —N(R⁸)CS—, —S(O)₂N(R⁸)—, —N(R⁸)O—, —ON(R⁸)—, —N(R⁸)N(R⁸)—, —N(R⁸)CON(R⁸)—, —N(R⁸)CSN(R⁸)—, or —N(R⁸)SO₂N(R⁸)— groups. Where the linker group contains two R⁸ substituents, these may be the same or different.

When R^{3a}, R⁴, R⁵, R⁶, R⁷ and/or R⁸ is present as a C₁₋₆alkyl group it may be a straight or branched C₁₋₆alkyl group, e.g. a C₁₋₃alkyl group such as a methyl, ethyl or i-propyl group. C₃₋₈cycloalkyl groups represented by R^{3a}, R⁴, R⁵, R⁶ and/or R⁷ include C₃₋₆cycloalkyl groups e.g. cyclopropyl, cyclobutyl, cyclopentyl and cyclohexyl groups. Optional substituents which may be present on such alkyl or cycloalkyl groups include for example one, two or three substituents which may be the same or different selected from halogen atoms, for example fluorine, chlorine, bromine or iodine atoms, or hydroxy or C₁₋₆alkoxy e.g. methoxy or ethoxy groups.

When the groups R⁵ and R⁶ or R⁶ and R⁷ are both C₁₋₆alkyl groups these groups may be joined, together with the N atom to which they are attached, to form a heterocyclic ring. Such heterocyclic rings may be optionally interrupted by a further heteroatom selected from —O—, —S— or —N(R⁵)—. Particular examples of such heterocyclic rings include piperidinyl, morpholinyl, thiomorpholinyl, pyrrolidinyl, imidazolidinyl and piperazinyl rings.

When Alk² is present as an optionally substituted aliphatic or heteroaliphatic chain it may be any optionally substituted aliphatic or heteroaliphatic chain as described hereinafter for Alk¹.

Halogen atoms represented by R⁴ in the optional Ar¹ substituents include fluorine, chlorine, bromine, or iodine atoms.

Examples of the substituents represented by -L³(Alk¹)ₜL⁴(R⁴)ᵤ when present in Ar¹ groups in compounds of the invention include atoms or groups -L³Alk²L⁴R⁴, -L³Alk²R⁴, -L³R⁴, —R⁴ and -Alk²R⁴ wherein L³, Alk², L⁴ and R⁴ are as defined above. Particular examples of such substituents include -L³CH₂L⁴R⁴, -L³CH(CH₃)L⁴R⁴, -L³(CH₂)₂L⁴R⁴, -L³CH₂R⁴, -L³CH(CH₃)R⁴, -L³(CH₂)₂R⁴, —CH₂R⁴, —CH(CH₃)R⁴, —(CH₂)₂R⁴ and —R⁴ groups.

Thus Ar¹ in compounds of the invention may be optionally substituted for example by one, two, three or more halogen atoms, e.g. fluorine, chlorine, bromine or iodine atoms, and/or C₁₋₆alkyl, e.g. methyl, ethyl, n-propyl, i-propyl, n-butyl or t-butyl, C₃₋₈cycloalkyl, e.g. cyclopropyl, cyclobutyl, cyclopentyl or cyclohexyl, C₁₋₆hydroxyalkyl, e.g. hydroxymethyl, hydroxyethyl or —C(OH)(CF₃)₂, carboxyC₁₋₆alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxypropylthio, C₁₋₆alkoxy, e.g. methoxy or ethoxy, hydroxyC₁₋₆alkoxy, e.g. 2-hydroxyethoxy, haloC₁₋₆alkyl, e.g. —CF₃, —CHF₂, —CH₂F, haloC₁₋₆alkoxy, e.g. —OCF₃, —OCHF₂, —OCH₂F, C₁₋₆alkylamino, e.g. methylamino or ethylamino, amino (—NH₂), aminoC₁₋₆alkyl, e.g. aminomethyl or aminoethyl, C₁₋₆dialkylamino, e.g. dimethylamino or diethylamino, C₁₋₆alkylaminoC₁₋₆alkyl, e.g. ethylaminoethyl, C₁₋₆dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆dialkylaminoC₁₋₆alkoxy, e.g. dimethylaminoethoxy, diethylaminoethoxy, diisopropylaminoethoxy or dimethylaminopropoxy, nitro, cyano, amidino, hydroxyl (—OH), formyl [HC(O)-], carboxyl (—CO₂H), —CO₂R⁵ e.g. —CO₂CH₃ or —CO₂C(CH₃)₃, C₁₋₆alkanoyl e.g. acetyl, thiol (—SH), thioC₁₋₆alkyl, e.g. thiomethyl or thioethyl, sulphonyl (—SO₃H), —SO₃R⁵, C₁₋₆alkylsulphinyl, e.g. methylsulphinyl, C₁₋₆ alkylsulphonyl, e.g. methylsulphonyl, aminosulphonyl (—SO₂NH₂), C₁₋₆alkylaminosulphonyl, e.g. methylaminosulphonyl or ethylaminosulphonyl, C₁₋₆dialkylaminosulphonyl, e.g. dimethylaminosulphonyl or diethylaminosulphonyl, phenylaminosulphonyl, carboxamido (—CONH₂), C₁₋₆alkylaminocarbonyl, e.g. methylaminocarbonyl or ethylaminocarbonyl, C₁₋₆dialkylaminocarbonyl, e.g. dimethylamino carbonyl or diethylamino carbonyl, aminoC₁₋₆alkylamino carbonyl, e.g. aminoethylaminocarbonyl, C₁₋₆alkylaminoC₁₋₆alkylaminocarbonyl, e.g. ethylaminoethylaminocarbonyl, C₁₋₆dialkylaminoC₁₋₆alkylaminocarbonyl, e.g. diethylaminoethylaminocarbonyl, aminocarbonylamino, C₁₋₆alkylaminocarbonylamino, e.g. methylaminocarbonylamino or ethylaminocarbonylamino, C₁₋₆dialkylaminocarbonylamino, e.g. dimethylaminocarbonylamino or diethylaminocarbonylamino, C₁₋₆alkylaminocabonylC₁₋₆alkylamino, e.g. methylaminocarbonylmethylamino, aminothiocarbonylamino, C₁₋₆alkylaminothio carbonylamino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothio carbonylC₁₋₆alkylamino, e.g. ethylaminothiocarbonylmethylamino, C₁₋₆alkylsulphonylamino, e.g. methylsulphonylamino or ethylsulphonylamino, C₁₋₆dialkylsulphonylamino, e.g. dimethylsulphonylamino or diethylsulphonylamino, aminosulphonylamino (—NHSO₂NH₂), C₁₋₆alkylaminosulphonylamino, e.g. methylaminosulphonylamino or ethylaminosulphonylamino, C₁₋₆dialkylaminosulphonylamino, e.g. dimethylaminosulphonylamino or diethylaminosulphonylamino, C₁₋₆alkanoylamino, e.g. acetylamino, aminoC₁₋₆alkanoylamino e.g. aminoacetylamino, C₁₋₆dialkylaminoC₁₋₆alkanoylamino, e.g. dimethylaminoacetylamino, C₁₋₆alkanoylaminoC₁₋₆alkyl, e.g. acetylaminomethyl, C₁₋₆alkanoylaminoC₁₋₆alkylamino, e.g. acetamidoethylamino, C₁₋₆alkoxycarbonylamino, e.g. methoxycarbonylamino, ethoxycarbonylamino or t-butoxycarbonylamino groups.

L² when present as part of the group R¹ in compounds of the invention may be a linker atom or group L²^{^{a}} or a linker (Alk³)L²^{^{a}}-, where Alk³ is an optionally substituted aliphatic or heteroaliphatic chain which may be any such chain as described hereinafter for Alk¹, and L²^{^{a}} may be any linker atom or group as described herein-before for L³.

Optionally substituted arylene groups represented by Ar² when present as part of the group R¹ include those aromatic groups as previously described for Ar¹.

Optionally substituted heteroarylene groups represented by Ar² when present as part of the group R¹ include those heteroaromatic groups as previously described for Ar¹.

Each divalent arylene or heteroarylene group represented by Ar² may be attached to the remainder of the molecule through any available ring carbon or nitrogen atoms.

The arylene and heteroarylene groups represented by Ar² may be optionally substituted by one, two or more substituents selected from the atoms or groups -L³(Alk²)ₜL⁴(R⁴)ᵤ described herein. Where two of these atoms or groups are present they may be the same or different.

When the group R² is present in compounds of the invention as a C₁₋₆alkyl group it may be for example a straight or branched C₁₋₆alkyl group e.g. a C₁₋₃alkyl group such as a methyl or ethyl group.

When the group R is present in R¹ in compounds of the invention as a derivative of a carboxylic acid it may be for example an acyclic or cyclic carboxylic acid ester or an amide. Particular acyclic esters and amides include CO₂Alk⁷ and CONR⁵R⁶ groups as defined herein. When R is a biostere of a carboxylic acid it may be for example a tetrazole or other acid such as phosphonic acid, phosphinic acid, sulphonic acid, sulphinic acid or boronic acid or an acylsulphonamide group.

Esters (—CO₂Alk⁷) and amide (—CONR⁵R⁶) derivatives of the carboxylic acid group (CO₂H) in compounds of Formula 1 may advantageously be used as prodrugs of the active compound. Such prodrugs are compounds which undergo biotransformation to the corresponding carboxylic acid prior to exhibiting their pharmacological effects and the invention particularly extends to prodrugs of the acids of Formula 1. Such prodrugs are well known in the art, see for example International Patent Application No. WO00/23419, Bodor, N. (Alfred Benzon Symposium, 1982, 17, 156-177), Singh, G. et al (J. Sci. Ind. Res., 1996, 55, 497-510) and Bundgaard, H., (Design of Prodrugs, 1985, Elsevier, Amsterdam).

Esterified carboxyl groups represented by the group CO₂Alk⁷ include groups wherein Alk⁷ is a straight or branched optionally substituted C₁₋₈alkyl group such as a methyl, ethyl, n-propyl, i-propyl, n-butyl, i-butyl, s-butyl, t-butyl, pentyl or neopentyl group; an optionally substituted C₂₋₈alkenyl group such as a propenyl e.g. 2-propenyl or butenyl e.g. 2-butenyl or 3-butenyl group, an optionally substituted C₂₋₈alkynyl group such as a ethynyl, propynyl e.g. 2-propynyl or butyryl e.g. 2-butyryl or 3-butyryl group, an optionally substituted C₃₋₈cycloalkyl group such as a cyclopropyl, cyclobutyl, cyclopentyl, cyclohexyl or cycloheptyl group; an optionally substituted C₃₋₈heterocycloalkyl group such as a tetrahydrofuranyl e.g. tetrahydrofuran-3-yl, pyrrolidinyl e.g. 1-methylpyrrolidinyl such as 1-methylpyrrolidin-3-yl, piperidinyl e.g. 1-methylpiperidinyl such as 1-methylpiperidin-4-yl, tetrahydropyranyl e.g. tetrahydropyran-4-yl or 2-oxo-[1,3]dioxol-4-yl e.g. 5-methyl-2-oxo-[1,3]dioxol-4-yl group; an optionally substituted C₃₋₈cycloalkylC₁₋₈alkyl group such as a cyclopentylmethyl, cyclohexylmethyl or cyclohexylethyl group; an optionally substituted C₃₋₈heterocycloalkylC₁₋₈alkyl group such as a morpholinyl-N-ethyl, thiomorpholinyl-N-methyl, pyrrolidinyl-N-ethyl, pyrrolidinyl-N-propyl, piperidinyl-N-ethyl, pyrazolidinyl-N-methyl or piperazinyl-N-ethyl group; an optionally substituted C₁₋₆alkyloxyC₁₋₆alkyl group such as a methyloxyethyl or propyloxyethyl group; an optionally substituted hydroxyC₁₋₆alkyl group such as a hydroxyethyl e.g. 2-hydroxyethyl or hydroxypropyl e.g. 2-hydroxypropyl, 3-hydroxypropyl or 2,3-dihydroxypropyl group; an optionally substituted C₁₋₆alkylthioC₁₋₆alkyl group such as an ethylthioethyl group; an optionally substituted C₁₋₆alkylsulfinylC₁₋₆alkyl group such as an methylsulfinylethyl group; an optionally substituted C₁₋₆alkylsulfonylC₁₋₆alkyl group such as an methylsulfonylmethyl group; an optionally substituted C₃₋₈cycloalkyloxyC₁₋₆alkyl group such as a cyclohexyloxymethyl group; an optionally substituted C₃₋₈cycloalkylthioC₁₋₆alkyl group such as a cyclopentylthiomethyl group; an optionally substituted C₃₋₈cycloalkylsulfinylC₁₋₆alkyl group such as a cyclopentyl-sulfinylmethyl group; an optionally substituted C₃₋₈cycloalkylsulfonylC₁₋₆alkyl group such as a cyclopentylsulfonylmethyl group; an optionally substituted C₁₋₆alkyloxycarbonylC₁₋₆alkyl group such as isobutoxy-carbonylpropyl group; an optionally substituted C₁₋₆alkyloxycarbonylC₁₋₆alkenyl group such as isobutoxycarbonylpentenyl group; an optionally substituted C₁₋₆alkyloxycarbonyloxyC₁₋₆alkyl group such as an ethyloxycarbonyloxymethyl or isopropoxycarbonyloxyethyl e.g 1-(isopropoxycarbonyloxy)ethyl or 2-(isopropoxycarbonyloxy)ethyl group; an optionally substituted C₁₋₆alkyloxycarbonyloxyC₁₋₆alkenyl group such as a isopropoxycarbonyloxybutenyl group, an optionally substituted C₃₋₈cycloalkyloxycarbonyloxyC₁₋₆alkyl group such as a cyclohexyloxycarbonyloxyethyl, e.g. a 2-(cyclohexyloxycarbonyloxy)ethyl group, an optionally substituted N-di-C₁₋₈alkylaminoC₁₋₈alkyl group such as a N-dimethylaminoethyl or N-diethylaminoethyl group; an optionally substituted N C₆₋₁₂aryl-NC₁₋₆alkylaminoC₁₋₆alkyl group such as a N-phenyl-N-methylaminomethyl group; an optionally substituted N-di-C₁₋₈alkylcarbamoylC₁₋₈alkyl group such as a N-diethylcarbamoylmethyl group; an optionally substituted C₆₋₁₂arylC₁₋₆alkyl group such as an optionally substituted benzyl, phenylethyl, phenylpropyl, 1-naphthylmethyl or 2-naphthylmethyl group; an optionally substituted heteroC₆₋₁₀arylC₁₋₆alkyl group, such as a pyridinylmethyl e.g. pyridin-4-ylmethyl or imidazolylethyl e.g. 2-imidazol-1-ylethyl group; a C₆₋₁₂aryl group such as an optionally substituted phenyl, 1-naphthyl or 2-naphthyl group; a C₆₋₁₂aryloxyC₁₋₈alkyl group such as an optionally substituted phenyloxymethyl, phenyloxyethyl, 1-naphthyloxymethyl, or 2-naphthyloxymethyl group; a C₆₋₁₂arylthioC₁₋₈alkyl group such as an optionally substituted phenylthioethyl group; a C₆₋₁₂arylsulfinylC₁₋₈alkyl group such as an optionally substituted phenyl-sulfinylmethyl group; a C₆₋₁₂arylsulfonylC₁₋₈alkyl group such as an optionally substituted phenylsulfonylmethyl group; an optionally substituted C₁₋₈alkanoyloxyC₁₋₈alkyl group, such as a acetoxymethyl, ethoxycarbonyloxyethyl, pivaloyloxymethyl, propionyloxyethyl or propionyloxypropyl group; an optionally substituted C₄₋₈imidoC₁₋₈alkyl group such as a succinimidomethyl or phthalamidoethyl group; a C₆₋₁₂aroyloxyC₁₋₈alkyl group such as an optionally substituted benzoyloxyethyl or benzoyloxypropyl group or a triglyceride such as a 2-substituted triglyceride e.g. a 1,3-di-C₁₋₈alkylglycerol-2-yl group such as a 1,3-diheptylglycerol-2-yl group. Optional substituents present on the Alk⁷ group include R¹³^{^{a}} substituents described below.

It will be appreciated that in the forgoing list of Alk⁷ groups the point of attachment to the remainder of the compound of Formula 1 is via the last described part of the Alk⁷ group. Thus, for example a methoxyethyl group would be attached by the ethyl group, whilst a morpholinyl-N-ethyl group would be attached via the N-ethyl group.

It will be further appreciated that in the forgoing list of Alk⁷ groups, where not specifically mentioned, alkyl groups may be replaced by alkenyl or alkynyl groups where such groups are as previously defined for Alk¹. Additionally these alkyl, alkenyl or alkynyl groups may optionally be interrupted by one, two or three linker atoms or groups where such linker atoms and groups are as previously defined for L³.

Further prodrugs of compounds of Formula 1 include cyclic esters where X is a —N(R²)— group in which R² becomes a C₁₋₆alkyl joining chain, especially a —CH₂— or —CH₂CH₂— chain, which is also connected to the acid group R to form a cyclic ester of Formula (1a):

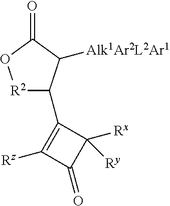

When present in the group R^{x}, R^{y} and/or R^{z} in compounds of Formula 1 the linker atom or group represented by L¹ may be any linker atom or group as described above for the linker atom or group L³. In addition L¹ may also be a Se atom.

When Alk¹ is present in the group R^{x}, R^{y} and/or R^{z} in compounds of Formula 1 as an optionally substituted aliphatic chain it may be an optionally substituted C₁₋₁₀aliphatic chain. Particular examples include optionally substituted straight or branched chain C₁₋₆alkylene, C₂₋₆alkenylene or C₂₋₆alkynylene chains.

Particular examples of aliphatic chains represented by Alk¹ include optionally substituted CH₂—, —(CH₂)₂—, —CH(CH₃)CH₂—, —(CH₂)₂CH₂—, —(CH₂)₃CH₂—, —CH(CH₃)(CH₂)₂—, —CH₂CH(CH₃)CH₂—, —C(CH₃)₂CH₂—, —CH₂C(CH₃)₂CH₂—, —(CH₂)₂C(CH₃)₂CH₂—, —(CH₂)₄CH₂—, —(CH₂)₅CH₂—, —CHCH—, —CHCHCH₂₋₁—CH₂CHCH—, —CHCHCH₂CH₂—, —CH₂CHCHCH₂—, —(CH₂)₂CHCH—, —CC—, —CCCH₂—, —CH₂CC—, —CCCH₂CH₂—, —CH₂CCCH₂— or —(CH₂)₂CC— chains.

Heteroaliphatic chains represented by Alk¹ when present in the group R^{x}, R^{y} and/or R^{z} in compounds of Formula 1 include the aliphatic chains just described for Alk¹ but with each additionally containing one, two, three or four heteroatoms or heteroatom-containing groups. Particular heteroatoms or groups include atoms or groups L⁵ where L⁵ is as defined above for L³ when L³ is a linker atom or group. Each L⁵ atom or group may interrupt the aliphatic chain, or may be positioned at its terminal carbon atom to connect the chain to an adjoining atom or group. Particular examples include optionally substituted —CH₂L⁵-, —CH₂CH₂L⁵-, -L⁵CH₂—, -L⁵CH₂CH₂—, -L⁵CH(CH₃)CH₂—, -L⁵CH₂CH(CH₃)CH₂—, L⁵CH₂CH₂CH(CH₃)—, -L⁵C(CH₃)₂CH₂—, —CH₂L⁵CH₂CH₂—, —(CH₂)₂L⁵CH₂—, —(CH₂)₃L⁵CH₂—, -L⁵(CH₂)₃—, -L⁵(CH₂)₄—, —CH₂L⁵CH₂CH₂L⁵CH₂— and —(CH₂)₂L⁵CH₂CH₂— chains.

The optional substituents which may be present on aliphatic or heteroaliphatic chains represented by Alk¹ include one, two, three or more substituents where each substituent may be the same or different and is selected from halogen atoms, e.g. fluorine, chlorine, bromine or iodine atoms, or —OH, —CO₂H, —CO₂R⁹, where R⁹ is an optionally substituted straight or branched C₁₋₆alkyl group as defined above for R⁴, —CONHR⁹, —CON(R⁹)₂, —COR⁵, e.g. —COCH₃, C₁₋₆alkoxy, e.g. methoxy or ethoxy, thiol, —S(O)R⁹, —S(O)₂R⁹, C₁₋₆alkylthio e.g. methylthio or ethylthio, amino or substituted amino groups. Substituted amino groups include —NHR⁹ and —N(R⁹)₂ groups. Where two R⁹ groups are present in any of the above substituents these may be the same or different.

Optionally substituted cycloaliphatic groups represented by the group R³ when present in the group R^{x}, R^{y} and/or R^{z} in compounds of the invention include optionally substituted C₃₋₁₀cycloaliphatic groups. Particular examples include optionally substituted C₃₋₁₀cycloalkyl, e.g. C₃₋₈cycloalkyl or C₃₋₁₀cycloalkenyl, e.g C₃₋₈cycloalkenyl groups.

Optionally substituted heterocycloaliphatic groups represented by the group R³ when present in the group R^{x}, R^{y} and/or R^{z} include optionally substituted C₃₋₁₀heterocycloaliphatic groups. Particular examples include optionally substituted C₃₋₁₀heterocycloalkyl, e.g. C₃₋₇ heterocycloalkyl, or C₃₋₁₀heterocycloalkenyl, e.g. C₃₋₇ hetercycloalkenyl groups, each of said groups containing one, two, three or four heteroatoms or heteroatom-containing groups L⁵ as defined above.

Optionally substituted polycycloaliphatic groups represented by the group R³ when present in the group R^{x}, R^{y} and/or R^{z} include optionally substituted C₇₋₁₀ bi- or tricycloalkyl or C₇₋₁₀ bi- or tricycloalkenyl groups. Optionally substituted heteropolycycloaliphatic groups represented by the group R³ include the optionally substituted polycycloaliphatic groups just described, but with each group additionally containing one, two, three or four L⁵ atoms or groups.

Particular examples of cycloaliphatic, polycycloaliphatic, heterocycloaliphatic and heteropolycycloaliphatic groups represented by the group R³ include optionally substituted cyclopropyl, cyclobutyl, cyclopentyl, cyclohexyl, cycloheptyl, cyclooctyl, cyclopropenyl, cyclobutenyl, cyclopentenyl, cyclohexenyl, cycloheptenyl, cyclooctenyl, 2-cyclobuten-1-yl, 2-cyclopenten-1-yl, 3-cyclopenten-1-yl, adamantyl, norbornyl, norbornenyl, tetrahydrofuranyl, tetrahydrothiophenyl, tetrahydrothiophene-1-oxide, tetrahydrothiophene-1,1-dioxide, pyrroline, e.g. 2- or 3-pyrrolinyl, pyrrolidinyl, pyrrolidinone, oxazolidinyl, oxazolidinone, dioxolanyl, e.g. 1,3-dioxolanyl, imidazolinyl, e.g. 2-imidazolinyl, imidazolidinyl, pyrazolinyl, e.g. 2-pyrazolinyl, pyrazolidinyl, pyranyl, e.g. 2- or 4-pyranyl, tetrahydropyranyl, tetrahydrothiopyranyl, tetrahydrothiopyran-1-oxide, tetrahydrothiopyran-1,1-dioxide, piperidinyl, piperidinone, dioxanyl e.g. 1,3-dioxanyl or 1,4-dioxanyl, morpholinyl, morpholinone, dithianyl, e.g. 1,3-dithianyl or 1,4-dithianyl, thiomorpholinyl, piperazinyl, 1,3,5-trithianyl, oxazinyl, e.g. 2H-1,3-, 6H-1,3-, 6H-1,2-, 2H-1,2- or 4H-1,4-oxazinyl, 1,2,5-oxathiazinyl, isoxazinyl, e.g. o- or p-isoxazinyl, oxathiazinyl, e.g. 1,2,5 or 1,2,6-oxathiazinyl, or 1,3,5,-oxadiazinyl groups.

The optional substituents which may be present on the cycloaliphatic, polycycloaliphatic, heterocycloaliphatic or heteropolycycloaliphatic groups represented by the group R³ include one, two, three or more substituents each selected from halogen atoms, e.g. fluorine, chlorine, bromine or iodine atoms, or C₁₋₆alkyl, e.g. methyl, ethyl, propyl or i-propyl, haloC₁₋₆alkyl, e.g. halomethyl or haloethyl such as difluoromethyl or trifluoromethyl, optionally substituted by hydroxyl, e.g. —C(OH)(CF₃)₂, C₁₋₆alkoxy, e.g. methoxy, ethoxy or propoxy, haloC₁₋₆alkoxy, e.g. halomethoxy or haloethoxy such as difluoromethoxy or trifluoromethoxy, thiol, C₁₋₆alkylthio e.g. methylthio, ethylthio or propylthio, or -(Alk⁴)_{g}R¹^{⁰} groups in which Alk⁴ is straight or branched C₁₋₃alkylene chain, g is zero or an integer 1 and R¹^{⁰} is a —OH, —SH, —N(R¹¹)₂, (in which R¹¹ is an atom or group as defined herein for R⁷)—CN, —CO₂R¹¹, —NO₂, —CON(R¹¹)₂, —CSN(R¹¹)₂, —COR¹¹, —CSN(R¹¹)₂, —N(R¹¹)COR¹¹, —N(R¹¹)CSR¹¹, —SO₂N(R¹¹)₂, —N(R¹¹)SO₂R¹¹, —N(R¹¹)CON(R¹)₂, —N(R¹¹)CSN(R¹¹), N(R¹¹)SO₂N(R¹¹)₂ or optionally substituted phenyl group. Where two R¹¹ atoms or groups are present in these substituents these may be the same or different or joined to form a heterocyclic ring as previously described when R⁵ and R⁶ are joined together. Optionally substituted phenyl groups include phenyl substituted by one, two or three of the R¹³ groups described below.

Additionally, when the group R³ is a heterocycloaliphatic group containing one or more nitrogen atoms each nitrogen atom may be optionally substituted by a group -(L⁶)ₚ(Alk⁵)_{q}R¹² in which L⁶ is —C(O)—, —C(O)O—, —C(S)—, —S(O)₂, —CON(R⁸)—, —CSN(R⁸)— or SO₂N(R⁸)—; p is zero or an integer 1; Alk⁵ is an optionally substituted aliphatic or heteroaliphatic chain; q is zero or an integer 1; and R¹² is a hydrogen atom or an optionally substituted cycloaliphatic, heterocycloaliphatic, polycycloaliphatic, polyheterocycloaliphatic, aromatic or heteroaromatic group.

C₁₋₃alkylene chains represented by Alk⁴ include CH₂, —CH₂CH₂, —CH₂CH₂CH₂—, —CH(CH₃)CH₂— and —CH₂CH(CH₃)— chains.

Optionally substituted aliphatic or heteroaliphatic chains represented by Alk⁵ include those optionally substituted chains described above for Alk¹. Optional substituents which may be present on these groups include those described above in relation to Alk¹.

Cycloaliphatic, heterocycloaliphatic, polycycloaliphatic or polyheterocycloaliphatic groups represented by R¹² include those groups just described for the group R³. Optional substituents which may be present on those groups include those described above in relation to R³ cycloaliphatic groups.

Aromatic or heteroaromatic groups represented by R¹² include those groups described herein for the group Ar¹. Optional substituents which may be present on these groups include those R¹³ optional substituents described hereinafter.

When the group R³ is an optionally substituted aromatic or heteroaromatic group it may be for example an aromatic or heteroaromatic group as described herein for the group Ar¹.

Optional substituents which may be present on the aromatic or heteroaromatic groups represented by the group R³ include one, two, three or more substituents, each selected from an atom or group R¹³ in which R¹³ is —R¹³^{^{a}} or -Alk⁶(R)ₘ, where R¹³^{^{a}} is a halogen atom, or an amino (—NH₂), substituted amino, nitro, cyano, amidino, hydroxyl (—OH), substituted hydroxyl, formyl, carboxyl (—CO₂H), esterified carboxyl, thiol (—SH), substituted thiol, —COR¹⁴ [where R¹⁴ is an -Alk⁶(R¹³^{^{a}})ₘ, aryl or heteroaryl group], —CSR¹⁴, —SO₃H, —SOR₁₄, —SO₂R¹⁴, —SO₃R¹⁴, —SO₂NH₂, —SO₂NHR¹⁴, SO₂N(R¹⁴)₂, —CONH₂, —CSNH₂, —CONHR¹⁴, —CSNHR¹⁴, —CON[R¹⁴]₂, —CSN(R¹⁴)₂, —N(R¹¹)SO₂R¹⁴, —N(SO₂R¹⁴)₂, —NH(R¹¹)SO₂NH₂, —N(R¹¹)SO₂NHR¹⁴, —N(R¹¹)SO₂N(R¹⁴)₂, —N(R¹¹)COR¹⁴, —N(R¹¹)CONH₂, —N(R¹¹)CONHR¹⁴, —N(R¹¹)CON(R¹⁴)₂, —N(R¹¹)CSNH₂, N(R¹¹)CSNHR¹⁴, —N(R¹¹)CSN(R¹⁴)₂, —N(R¹¹)CSR¹⁴, —N(R¹¹)C(O)OR¹⁴, —SO₂NHet¹ [where —NHet¹ is an optionally substituted C₅₋₇cyclicamino group optionally containing one or more other —O— or —S— atoms or N(R¹¹)—, —C(O)—, —C(S)—, S(O) or —S(O)₂ groups], —CONHet¹, —CSNHet¹, —N(R¹¹)SO₂NHet¹, —N(R¹¹)CONHet¹, —N(R¹¹)CSNHet¹, —SO₂N(R¹¹)Het² [where Het² is an optionally substituted monocyclic C₅₋₇carbocyclic group optionally containing one or more —O— or —S— atoms or —N(R¹¹)—, —C(O)— or —C(S)— groups], -Het², —CON(R¹¹)Het², —CSN(R¹¹)Het², —N(R¹¹)CON(R¹¹)Het², —N(R¹¹)CSN(R¹¹)Het², aryl or heteroaryl group; Alk⁶ is a straight or branched C₁₋₆alkylene, C₂₋₆alkenylene or C₂₋₆alkynylene chain, optionally interrupted by one, two or three —O— or —S— atoms or —S(O)ₙ [where n is an integer 1 or 2] or —N(R¹⁵)— groups [where R¹⁵ is a hydrogen atom or C₁₋₆alkyl, e.g. methyl or ethyl group]; and m is zero or an integer 1, 2 or 3. It will be appreciated that when two R¹¹ or R¹⁴ groups are present in one of the above substituents, the R¹¹ or R¹⁴ groups may be the same or different.

When in the group -Alk⁶(R¹³^{^{a}})ₘ m is an integer 1, 2 or 3, it is to be understood that the substituent or substituents R¹³^{^{a}} may be present on any suitable carbon atom in -Alk⁶. Where more than one R¹³^{^{a}} substituent is present these may be the same or different and may be present on the same or different atom in -Alk⁶. Clearly, when m is zero and no substituent R¹³^{^{a}} is present the alkylene, alkenylene or alkynylene chain represented by Alk⁶ becomes an alkyl, alkenyl or alkynyl group.

When R¹³^{^{a}} is a substituted amino group it may be for example a group —NHR¹⁴ [where R¹⁴ is as defined above] or a group —N(R¹⁴)₂ wherein each R¹⁴ group is the same or different.

When R¹³^{^{a}} is a halogen atom it may be for example a fluorine, chlorine, bromine, or iodine atom.

When R¹³^{^{a}} is a substituted hydroxyl or substituted thiol group it may be for example a group —OR¹⁴ or a —SR¹⁴ or —SC(═NH)NH₂ group respectively.

Esterified carboxyl groups represented by the group R^{13a} include groups of formula —CO₂Alk⁸ wherein Alk⁸ is a straight or branched, optionally substituted C₁₋₈alkyl group such as a methyl, ethyl, n-propyl, 1-propyl, n-butyl, i-butyl, s-butyl or t-butyl group; a C₆₋₁₂arylC₁₋₈alkyl group such as an optionally substituted benzyl, phenylethyl, phenylpropyl, 1-naphthylmethyl or 2-naphthylmethyl group; a C₆₋₁₂aryl group such as an optionally substituted phenyl, 1-naphthyl or 2-naphthyl group; a C₆₋₁₂aryloxyC₁₋₈alkyl group such as an optionally substituted phenyloxymethyl, phenyloxyethyl, 1-naphthyloxymethyl, or 2-naphthyloxymethyl group; an optionally substituted C₁₋₈alkanoyloxyC₁₋₈alkyl group, such as a pivaloyloxymethyl, propionyloxyethyl or propionyloxypropyl group; or a C₆₋₁₂aroyloxyC₁₋₈alkyl group such as an optionally substituted benzoyloxyethyl or benzoyloxypropyl group. Optional substituents present on the Alk⁸ group include R^{13a} substituents described above.

When Alk⁶ is present in or as a substituent it may be for example a methylene, ethylene, n-propylene, i-propylene, n-butylene, i-butylene, s-butylene, t-butylene, ethenylene, 2-propenylene, 2-butenylene, 3-butenylene, ethynylene, 2-propynylene, 2-butynylene or 3-butynylene chain, optionally interrupted by one, two, or three —O— or —S—, atoms or —S(O)—, —S(O)₂— or —N(R⁸)— groups.

Aryl or heteroaryl groups represented by the groups R¹³^{^{a}} or R¹⁴ include mono- or bicyclic optionally substituted C₆₋₁₂aromatic or C₁₋₉heteroaromatic groups as described above for the group Ar¹. The aromatic and heteroaromatic groups may be attached to the remainder of the compound of Formula 1 by any carbon or hetero e.g. nitrogen atom as appropriate.

When —NHet¹ or -Het² forms part of a substituent R¹³ each may be for example an optionally substituted pyrrolidinyl, pyrazolidinyl, piperazinyl, morpholinyl, thiomorpholinyl, piperidinyl or thiazolidinyl group. Additionally Het² may represent for example, an optionally substituted cyclopentyl or cyclohexyl group. Optional substituents which may be present on —NHet¹ or -Het² include those optional substituents described above in relation to aliphatic chains represented by Alk¹.

Particularly useful atoms or groups represented by R¹³ include fluorine, chlorine, bromine or iodine atoms, or C₁₋₆alkyl, e.g. methyl, ethyl, n-propyl, i-propyl, n-butyl or t-butyl, optionally substituted phenyl, pyridyl, pyrimidinyl, pyrrolyl, furyl, thiazolyl, thienyl, morpholinyl, thiomorpholinyl, piperazinyl, e.g. t-butyloxycarbonylpiperazinyl, pyrrolidinyl, dioxolanyl, dioxanyl, oxazolidinyl, thiazolidinyl, imidazolidinyl or piperidinyl, C₁₋₆hydroxyalkyl, e.g. hydroxymethyl or hydroxyethyl, carboxyC₁₋₆alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxypropylthio, C₁₋₆alkoxy, e.g. methoxy or ethoxy, hydroxyC₁₋₆alkoxy, e.g. 2-hydroxyethoxy, optionally substituted phenoxy, pyridyloxy, thiazolyoxy, phenylthio or pyridylthio, C₄₋₇cycloalkyl, e.g. cyclobutyl, cyclopentyl, C₅₋₇cycloalkoxy, e.g. cyclopentyloxy, haloC₁₋₆alkyl, e.g. trifluoromethyl, haloC₁₋₆alkoxy, e.g. trifluoromethoxy, C₁₋₆alkylamino, e.g. methylamino, ethylamino or propylamino, C₆₋₁₂arylC₁₋₆alkylamino, e.g. benzylamino, 4-fluorobenzylamino or 4-hydroxyphenylethylamino, amino (—NH₂), aminoC₁₋₆alkyl, e.g. aminomethyl or aminoethyl, C₁₋₆dialkylamino, e.g. dimethylamino or diethylamino, aminoC₁₋₆alkylamino, e.g. amino ethylamino or aminopropylamino, optionally substituted Het¹NC₁₋₆alkylamino, e.g. 3-morpholinopropylamino, C₁₋₆alkylaminoC₁₋₁₆alkyl, e.g. ethylaminoethyl, C₁₋₆dialkylaminoC₁₋₆alkyl, e.g. diethylaminoethyl, aminoC₁₋₆alkoxy, e.g. amino ethoxy, C₁₋₆alkylaminoC₁₋₆alkoxy, e.g. methylaminoethoxy, C₁₋₆dialkylaminoC₁₋₆alkoxy, e.g. dimethylaminoethoxy, diethytaminoethoxy, diisopropylaminoethoxy, or dimethylaminopropoxy, hydroxyC₁₋₆alkylamino, e.g. 2-hydroxyethylamino, 3-hydroxypropylamino or 3-hydroxybutylamino, imido, such as phthalimido or naphthalimido, e.g. 1,8-naphthalimido, nitro, cyano, amidino, hydroxyl (—OH), formyl[HC(O)—], carboxyl (—CO₂H), —CO₂Alk⁸ [where Alk⁸ is as defined above], C₁₋₆alkanoyl e.g. acetyl, propyryl or butyryl, optionally substituted benzoyl, thiol (—SH), thioC₁₋₆alkyl, e.g. thiomethyl or thioethyl, —SC(═NH)NH₂, sulphonyl (—SO₃H), —SO₃Alk⁸, C₁₋₆alkylsulphinyl, e.g. methylsulphinyl, ethylsulphinyl or propylsulphinyl, C₁₋₆alkylsulphonyl, e.g. methylsulphonyl, ethylsulphonyl or propylsulphonyl, aminosulphonyl (—SO₂NH₂), C₁₋₆alkylaminosulphonyl, e.g. methylaminosulphonyl, ethylaminosulphonyl or propylaminosulphonyl C₁₋₆dialkylaminosulphonyl, e.g. dimethylaminosulphonyl or diethylaminosulphonyl, phenylaminosulphonyl, carboxamido (—CONH₂), C₁₋₆alkylaminocarbonyl, e.g. methylaminocarbonyl, ethylaminocarbonyl or propylaminocarbonyl, C₁₋₆dialkylaminocarbonyl, e.g. dimethylaminocarbonyl or diethylaminocarbonyl, aminoC₁₋₅alkylaminocarbonyl, e.g. aminoethylaminocarbonyl, C₁₋₆alkylaminoC₁₋₆alkylaminocarbonyl, e.g. methylaminoethylaminocarbonyl, C₁₋₆dialkylaminoC₁₋₆alkylaminocarbonyl, e.g. diethylaminoethylaminocarbonyl, aminocarbonylamino, C₁₋₆alkylaminocarbonylamino, e.g. methylaminocarbonylamino or ethylaminocarbonylamino, C₁₋₆dialkylaminocarbonylamino, e.g. dimethylaminocarbonylamino or diethylaminocarbonylamino, C₁₋₆alkylaminocabonylC₁₋₆alkylamino, e.g. methylaminocarbonylmethylamino, aminothiocarbonylamino, C₁₋₆alkylaminothiocarbonylamino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothiocarbonylC₁₋₆alkylamino, e.g. ethylaminothiocarbonylmethylamino, CONHC(═NH)NH₂, C₁₋₆alkylsulphonylamino, e.g. methylsulphonylamino or ethylsulphonylamino, haloC₁₋₆alkylsulphonylamino, e.g. trifluoromethylsulphonylamino, C₁₋₆dialkylsulphonylamino, e.g. dimethylsulphonylamino or diethylsulphonylamino, optionally substituted phenylsulphonylamino, aminosulphonylamino (NHSO₂NH₂), C₁₋₆alkylaminosulphonylamino, e.g. methylaminosulphonylamino or ethylaminosulphonylamino, C₁₋₆dialkylaminosulphonylamino, e.g. dimethylaminosulphonylamino or diethylaminosulphonylamino, optionally substituted morpholinesulphonylamino or morpholinesulphonylC₁₋₆alkylamino, optionally substituted phenylaminosulphonylamino, C₁₋₆alkanoylamino, e.g. acetylamino, aminoC₁₋₆alkanoylamino e.g. aminoacetylamino, C₁₋₆dialkylaminoC₁₋₆alkanoylamino, e.g. dimethylaminoacetylamino, C₁₋₆alkanoylaminoC₁₋₆alkyl, e.g. acetylaminomethyl, C₁₋₆alkanoylaminoC₁₋₆alkylamino, e.g. acetamidoethylamino, C₁₋₆alkoxycarbonylamino, e.g. methoxycarbonylamino, ethoxycarbonylamino or t-butoxycarbonylamino or optionally substituted benzyloxy, pyridylmethoxy, thiazolylmethoxy, benzyloxycarbonylamino, benzyloxycarbonylaminoC₁₋₆alkyl e.g. benzyloxycarbonylaminoethyl, thiobenzyl, pyridylmethylthio or thiazolylmethylthio groups.

Where desired, two R¹³ substituents may be linked together to form a cyclic group such as a cyclic ether, e.g. a C₁₋₆alkylenedioxy group such as methylenedioxy or ethylenedioxy.

It will be appreciated that where two or more R¹³ substituents are present, these need not necessarily be the same atoms and/or groups. In general, the substituent(s) may be present at any available ring position in the aromatic or heteroaromatic group represented by R³.

When the groups R^{x} and R^{y} are joined together to form an optionally substituted spiro linked cycloaliphatic or heterocycloaliphatic group joined to the cyclobutenone ring as defined by Formula 1 it may be any such cycloaliphatic or heterocycloaliphatic group as previously described for R³. Optional substituents which may be present on such spiro linked cycloaliphatic or heteroaliphatic groups include those optional substituents as described in relation to R³.

In the compounds according to Formula 1, the group R¹ is preferably an Ar¹L²Ar²Alk-group. In compounds of this type Ar¹ is preferably an optionally substituted phenyl, monocyclic heteroaromatic or bicyclic heteroaromatic group. Particularly useful monocyclic heteroaromatic groups are optionally substituted five- or six-membered heteroaromatic groups as described previously, especially five- or six-membered heteroaromatic groups containing one or two heteroatoms selected from oxygen, sulfur or nitrogen atoms. Nitrogen-containing groups are especially useful, particularly pyridyl or pyrimidinyl groups. Particularly useful substituents present on these monocyclic Ar¹ groups include halogen atoms or alkyl, haloalkyl, —OR⁵, —SR⁵—NR⁵R⁶, —CO₂H, —CO₂CH₃, —NO₂, —N(R⁵)COR⁶ or —CN groups as described above in relation to the compounds of Formula 1. Particularly useful bicyclic heteroaromatic groups represented by Ar¹ include optionally substituted ten-membered fused-ring heteroaromatic groups containing one, two or three, especially one or two heteroatoms, especially nitrogen atoms. Particular examples include optionally substituted naphthyridinyl, especially 2,6-naphthyridinyl, 2,7-naphthyridinyl, quinolinyl and isoquinolinyl, especially isoquinolin-1-yl groups. Particular optional substituents include those just described for monocyclic heteroaromatic groups. Additionally, in compounds according to the invention X is preferably an —N(R²)— group and V is preferably an oxygen atom.

The presence of certain substituents in the compounds of Formula 1 and Formula 2 may enable salts of the compounds to be formed. Suitable salts include pharmaceutically acceptable salts, for example acid addition salts derived from inorganic or organic acids, and salts derived from inorganic and organic bases.

Acid addition salts include hydrochlorides, hydrobromides, hydroiodides, alkylsulphonates, e.g. methanesulphonates, ethanesulphonates, or isothionates, arylsulphonates, e.g. p-toluenesulphonates, besylates or napsylates, phosphates, sulphates, hydrogen sulphates, acetates, trifluoroacetates, propionates, citrates, maleates, fumarates, malonates, succinates, lactates, oxalates, tartrates and benzoates.

Salts derived from inorganic or organic bases include alkali metal salts such as sodium or potassium salts, alkaline earth metal salts such as magnesium or calcium salts, and organic amine salts such as morpholine, piperidine, dimethylamine or diethylamine salts.

Particularly useful salts of compounds according to the invention include pharmaceutically acceptable salts, especially acid addition pharmaceutically acceptable salts.

### SUMMARY

According to the present invention, schizophrenia in mammals is treated by the administration of a therapeutically effective amount of an α4 integrin inhibitor of Formula 1 or Formula 2 or a pharmaceutically acceptable salt, solvate, hydrate or N-oxide thereof.

Compounds of Formula 1 and/or Formula 2 and pharmaceutically acceptable salts, solvates, hydrates and N-oxides thereof may be made using synthetic methods known in the art. Methods of making such compounds are described in U.S. Pat. Nos. 7,501,437 and 6,835,738, which description is incorporated by reference herein in its entirety.

An embodiment of the invention is a composition for the treatment of schizophrenia comprising at least one compound of Formula 1 or a pharmaceutically acceptable salt, solvate, hydrate or N-oxide thereof, incorporated in a pharmaceutically acceptable adjuvant, excipient, diluent or carrier composition.

An embodiment of the invention is a composition for the treatment of schizophrenia comprising at least one compound of Formula 2 or a pharmaceutically acceptable salt, solvate, hydrate or N-oxide thereof, incorporated in a pharmaceutically acceptable adjuvant, excipient, diluent or carrier composition.

An embodiment of the invention is a method of treating schizophrenia in a mammal in need of such treatment comprising administering a therapeutically effective amount of a compound selected from the group consisting of (2S)-2-(2-Bromo-3-oxo-spiro[3.5]non-1-en-1-ylamino)-3-[4-([2,7]naphthyridin-1-ylamino)phenyl]proprionic acid and pharmaceutically acceptable salts, hydrates, solvates and N-oxides thereof.

Another embodiment of the invention comprises compositions used for treating schizophrenia comprising at least one compound selected from the group consisting of (2S)-2-(2-Bromo-3-oxo-spiro[3.5]non-1-en-1-ylamino)-3-[4-([2,7]naphthyridin-1-ylamino)phenyl]proprionic acid and pharmaceutically acceptable salts, hydrates, solvates and N-oxides thereof, incorporated in a pharmaceutically acceptable adjuvant, excipient, diluent, or carrier composition.

### DESCRIPTION OF THE FIGURES

FIG. 1 shows the reversal of PCP-induced pre-pulse-inhibition deficit in adult male rats by 50 mg/kg of (2S)-2-(2-Bromo-3-oxo-spiro[3.5]non-1-en-1-ylamino)-3-[4-([2,7]naphthyridin-1-ylamino)phenyl]proprionic acid p.o., at a pre-pulse intensity of 73 dB.

FIG. 2 shows the reversal of PCP-induced pre-pulse-inhibition deficit in adult male rats by 50 mg/kg of (2 S)-2-(2-Bromo-3-oxo-spiro[3.5]non-1-en-1-yl amino)-3-[4-([2,7]naphthyridin-1-ylamino)phenyl]proprionic acid p.o., as shown by an average of the inhibition found at all three pre-pulse intensities, 69, 73 and 81 dB.

### DETAILED DESCRIPTION

Embodiments of the invention provide methods for treating schizophrenia and/or symptoms of schizophrenia with one or more compounds of Formula 1 or Formula 2 and pharmaceutically acceptable salts, solvates, hydrates and N-oxides thereof. Symptoms of schizophrenia are well-documented by the literature and diagnostic authorities on schizophrenia and include delusions; hallucinations; movement and thought disorders, such as disorganized speech and/or grossly disorganized or catatonic behavior; social disturbances; flat affect; deficits in expressing emotion and experiencing pleasure such as alogia and/or avolition; disorganized thinking, slow thinking; difficulty understanding; poor concentration; poor memory; difficulty expressing thoughts; and difficulty integrating thoughts, feelings and behavior. A particular embodiment of the invention provides a method of treating positive symptoms of schizophrenia by administering to a human in need of schizophrenia treatment a therapeutically effective amount of an α4 integrin inhibitor of Formula 1 or Formula 2 or pharmaceutically acceptable salts, solvates, hydrates and N-oxides thereof.

Embodiments of the invention also provide methods for treating positive symptoms of schizophrenia in other psychotic diseases, for example, bipolar disorder, delusional disorder, psychotic depression, Tourette syndrome, autism spectrum disorder, OCD, dementia and Alzheimer's disease, with one or more compounds of Formula 1 or Formula 2 and pharmaceutically acceptable salts, solvates, hydrates and N-oxides thereof.

Embodiments of the invention provide methods for treating schizophrenia and/or symptoms of schizophrenia and/or positive symptoms of schizophrenia in other psychotic diseases, by administering to a subject in need of schizophrenia treatment a therapeutically effective amount of a compound of the genus of α4 integrin inhibitors described in U.S. Pat. No. 7,501,437 or U.S. Pat. No. 6,835,738. A particular embodiment of the invention provides a method of treating positive symptoms of schizophrenia by administering to a human in need of schizophrenia treatment a therapeutically effective amount of a compound of the genus of α4 integrin inhibitors described in U.S. Pat. No. 7,501,437 or U.S. Pat. No. 6,835,738.

According to embodiments of the invention, a therapeutically effective amount of a compound of Formula 1 or Formula 2 that modulates α4 integrin activity or expression is administered to a subject to treat schizophrenia and/or symptoms of schizophrenia and/or positive symptoms of schizophrenia in other psychotic diseases. The ability of any compound of Formula 1 or Formula 2 to modulate α4 integrin activity may be simply determined by employing tests such as those described in U.S. Pat. No. 7,501,437.

A compound useful in carrying out prophylactic and/or therapeutic method embodiments of the invention is advantageously formulated in a pharmaceutical composition in combination with one or more pharmaceutically acceptable carriers, excipients or diluents.

Pharmaceutical compositions useful in carrying out the invention may take a form suitable for oral, buccal, parenteral, nasal, topical or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium glycollate); or welting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles and preservatives. The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

The compounds for Formula 1 and Formula 2 may be formulated for parenteral administration by injection e.g. by bolus injection or infusion. Formulations for injection may be presented in unit dosage form, e.g. in glass ampoule or multi-dose containers, e.g. glass vials. The compositions for injection may take such forms as suspensions, solutions or emulsions in oily or aqueous vehicles, and may contain formulatory agents such as suspending, stabilizing, preserving and/or dispersing agents. Alternatively, the active ingredient may be in powder form for constitution with a suitable vehicle, e.g. sterile pyrogen-free water, before use.

In addition to the formulations described above, the compounds of Formula 1 and Formula 2 may also be formulated as a depot preparation. Such long acting formulations may be administered by implantation or by intramuscular injection.

For nasal administration or administration by inhalation, the compounds for use according to the present invention are conveniently delivered in the form of an aerosol spray presentation for pressurized packs or a nebuliser, with the use of suitable propellant, e.g. dichlorodifluoromethane, trichloro-fluoromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

The quantity of a compound of the invention required for the prophylaxis or treatment of schizophrenia and/or symptoms of schizophrenia will vary depending on the compound chosen, the condition of the patient to be treated, and the route of administration. In general, however, daily dosages may range from around 50 ng/kg to 100 mg/kg, e.g. around 0.1 mg/kg to 50 mg/kg, body weight for oral or buccal administration, from around 10 ng/kg to 50 mg/kg body weight for parenteral administration and around 50 ng to around 1000 mg, e.g. around 0.5 mg to around 1000 mg, for nasal administration or administration by inhalation or insufflation, although it will, of course, readily be understood that the amount of the compound actually to be administered will be determined by a physician, in the light of all the relevant circumstances including the condition to be treated, the choice of compound to be administered and the choice of route of administration. In another embodiment, a suitable dosage range of the active ingredient is from about 75 ng/kg to about 5 mg/kg of body weight taken at necessary intervals. In another embodiment, a suitable dosage range of the active ingredient is from about 2 mg to about 100 mg taken three times a day. In another embodiment, a suitable dosage range for human patients is from about 6 mg to about 300 mg, daily. The effective dosage range of the pharmaceutically acceptable esters, salts or other derivatives can be calculated based on the weight of the parent compound to be delivered.

In one embodiment, the dosage and administration are such that α4 integrin activity or expression is only partially modulated so as to avoid any unacceptably deleterious effects.

A further embodiment of the invention provides methods for treating schizophrenia and/or symptoms of schizophrenia and/or positive symptoms of schizophrenia in other psychotic diseases in a subject in need of such treatment by inhibiting α4 integrin by administering a therapeutically effective amount of at least one compound selected from the group consisting of (2S)-2-(2-Bromo-3-oxo-spiro[3.5]non-1-en-1-ylamino)-3-[4-([2,7]naphthyridin-1-ylamino)phenyl]proprionic acid and pharmaceutically acceptable salts, hydrates, solvates and N-oxides thereof.

Another embodiment of the invention comprises compositions used for treating schizophrenia and/or symptoms of schizophrenia and/or positive symptoms of schizophrenia in other psychotic diseases comprising at least one compound selected from the group consisting of (2S)-2-(2-Bromo-3-oxo-spiro[3.5]non-1-en-1-ylamino)-3-[4-([2,7]naphthyridin-1-ylamino)phenyl]proprionic acid and pharmaceutically acceptable salts, hydrates, solvates and N-oxides thereof incorporated in a pharmaceutically acceptable adjuvant, diluent, or carrier composition.

### Experimental Results

Prepulse inhibition of the acoustic startle response (PPI) is the reduction of the startle response to a sudden pulse of noise when it is preceded by a weak prepulse stimulus. The effect of the prepulse is considered as a form of sensorimotor gating and is common to many species, from mice to humans.

Deficits in PPI are commonly associated with schizophrenia or positive symptoms of schizophrenia in other psychotic diseases, and can be reproducibly induced in laboratory animals by administration of psychotropic agents such as dopamine agonists (apomorphine), NMDA antagonists (phencyclidine) or even serotonin agonists. These deficits can be partially reversed by antipsychotic agents such as Clozapine and Risperidone. The effectiveness of a number of putative antipsychotic agents in animal models of PPI has been shown to correlate closely to their clinical effectiveness. Given their predictive value, animal models of PPI are now one of the most widely-used preclinical models for evaluating novel antipsychotic drugs.

Methods:

Male Sprague-Dawley rats weighing 210-250 g at time of experiment were used. Animals were housed in a temperature and humidity controlled environment and allowed free access to food and water prior to use.

The amplitude of the inherent startle response varies considerably from animal to animal. The gain of the platforms must be calibrated to ensure that the average startle waveform falls within the desired range. To this end, baseline studies were conducted before the full PPI protocol to allow the calibration of the platforms to be fine-tuned to the startle waveform amplitude. This also allows the animals to become acclimatised to the testing equipment prior to drug administration.

Animals were placed in restraining holders and transferred to the designated chamber. Once all holders were securely fastened to the platform, the Baseline protocol was started. This consists of:
Acclimation Period: Animals were exposed to 5 minutes of 65 decibel (dB) background noise (white noise).
Block 1: 10× startle stimulus alone (white noise, 120 dB, 40 ms)
Block 2: 6× startle stimulus alone
   6× pre-pulse plus startle stimulus (pure tone, 16 dB above background noise,
   20 ms) followed 100 ms later by the startle stimulus.
During this time, the amplitude of the startle responses was recorded. On completion of this protocol, the sensitivity of the equipment was re-calibrated where necessary by adjusting the gain settings.

The same group of rats was again subjected to the Baseline protocol described above. Again, on completion of this protocol, the sensitivity of the equipment was re-calibrated where necessary by adjusting the gain settings. The gain settings required for each rat were noted for use in the subsequent PPI protocol. These animals were then returned to their home cages for subsequent dosing with compound.

The study compounds were suspended from dry powder stocks in Vehicle (5% Tween 80/5% PEG/saline) and sonicated for 60 minutes at 37° C. to generate suspensions suitable for oral dosing. Control rats received Vehicle by oral gavage. PCP was dissolved in sterile saline (0.9% NaCl).

Rats were dosed with either saline (0.9% NaCl) or 2.5 mg/kg PCP by intraperitoneal injection 15 minutes prior to PPI testing.

(2 S)-2-(2-Bromo-3-oxo-spiro[3.5]non-1-en-1-ylamino)-3-[4-([2,7]naphthyridin-1-ylamino)phenyl]proprionic acid or Clozapine was administered orally by gavage 45 minutes before challenge with PCP or saline. (2S)-2-(2-Bromo-3-oxo-spiro[3.5]non-1-en-1-ylamino)-3-[4-([2,7]naphthyridin-1-ylamino)phenyl]proprionic acid was administered at 10 mg/kg and 50 mg/kg. Clozapine was administered at 5 mg/kg. Vehicle control rats received Vehicle (5% Tween 80/5% PEG/saline) by oral gavage 45 minutes before challenge with PCP or saline.

Exactly 15 minutes after the injection of PCP or saline, the full PPI protocol was started. Animals were exposed to 5 minutes of 65 dB background noise (white noise) as an acclimation period, before the acoustic stimuli began.
Block 1: 6× startle stimulus alone (white noise, 120 dB, 40 ms)
Block 2: 14× startle stimulus
   8× no pulse
   30× pre-pulse (10 each of 4, 8, and 16 dB above the background noise) followed 100 ms later by the startle stimulus. This corresponds to intensities of 69, 73 and 81 dB, respectively. A total of 52 trials were presented to the animals in this block in a pseudo-random order with a variable interval of 8 to 23 seconds, averaging 15 s between trials).
Block 3: 6× startle stimulus alone

Pre-pulse inhibition was calculated from trials in Block 2 by the following formula:

% PPI=(100×[(pulse alone score)−(prepulse+pulse score)]/pulse alone

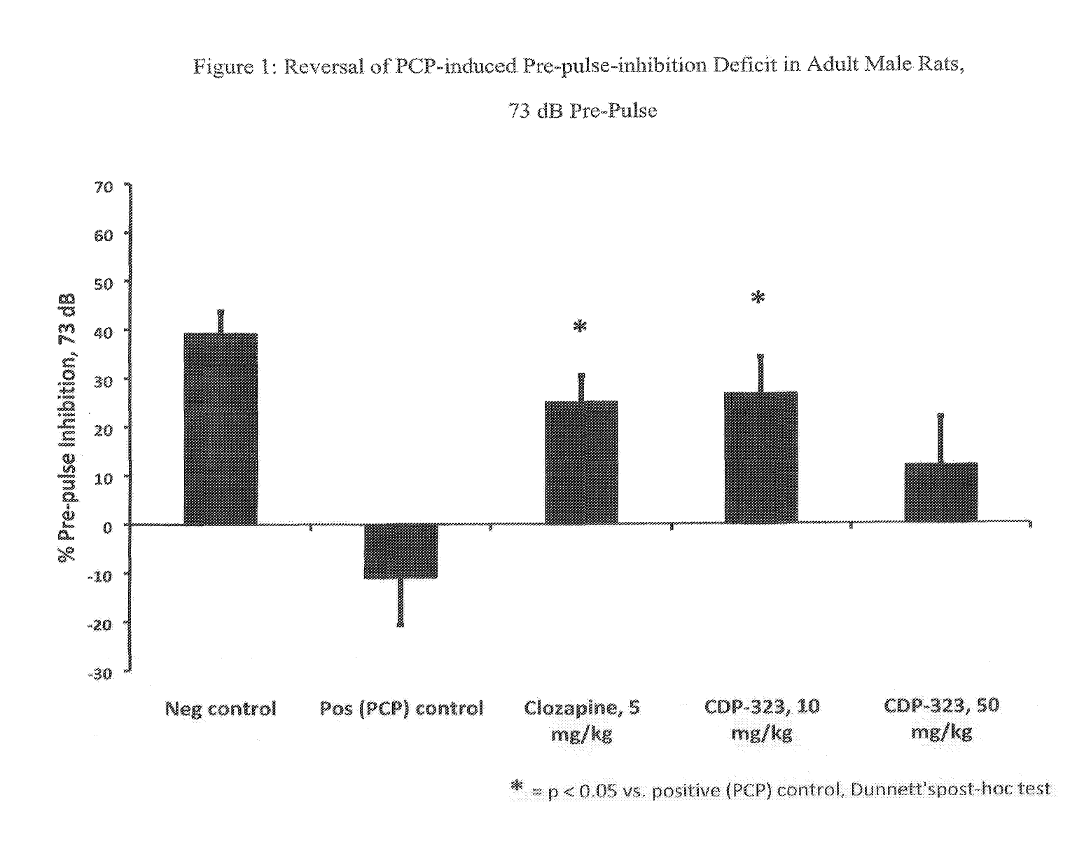

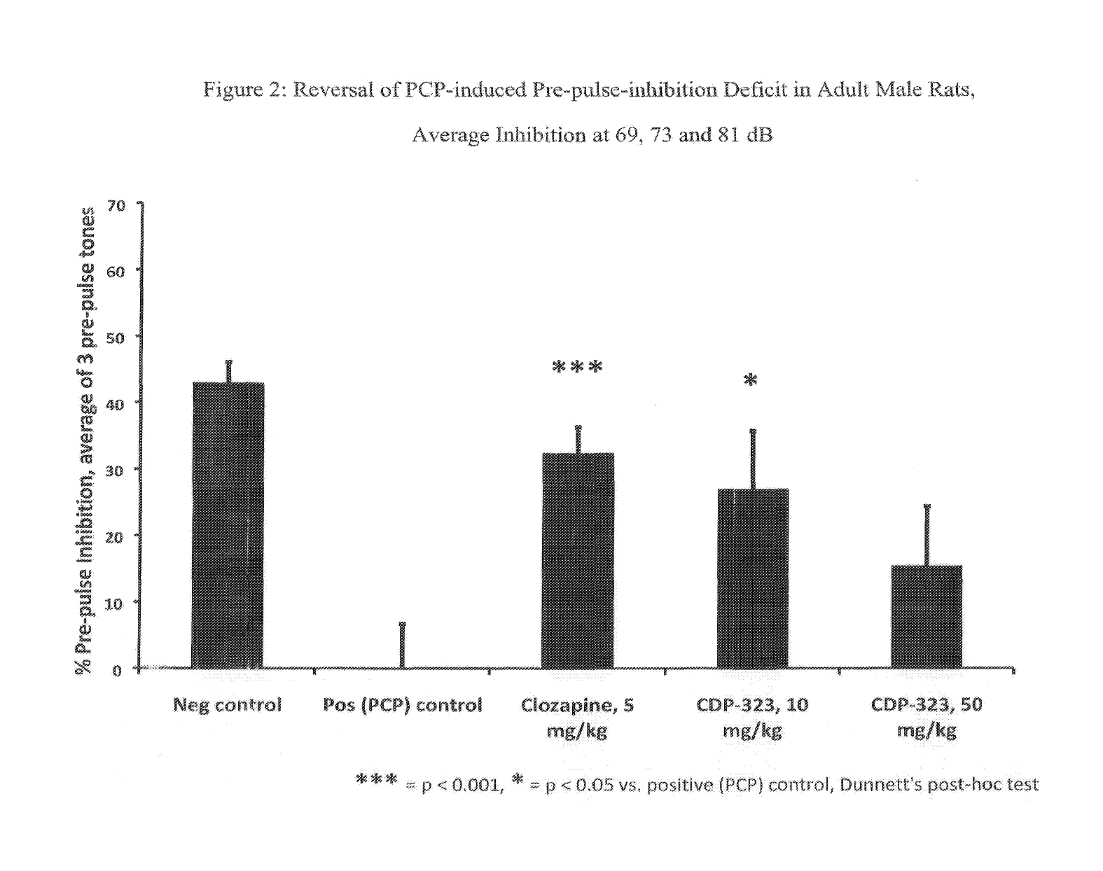

At the lowest and highest prepulse intensities of 69 and 81 dB (4 and 16 dB above background noise), in this experiment, (2S)-2-(2-Bromo-3-oxo-spiro[3.5]non-1-en-1-ylamino)-3-[4-([2,7]naphthyridin-1-ylamino)phenyl]proprionic acid did not have a statistically significant effect on pre-pulse inhibition. When the prepulse intensity was 73 dB (8 dB above background noise), the ability of (2 S)-2-(2-Bromo-3-oxo-spiro[3.5]non-1-en-1-ylamino)-3-[4-([2,7]naphthyridin-1-ylamino)phenyl]proprionic acid to attenuate the PCP-induced deficit in PPI was more evident. See FIG. 1. At a dose of 50 mg/kg, MGCD-0103 significantly (p<0.05) reversed the PCP-induced deficit in PPI. Furthermore, when the percent pre-pulse inhibition was averaged for all three prepulse intensities (69, 73 and 81 dB), at a dose of 50 mg/kg, MGCD-0103 significantly (p<0.05) reversed the PCP-induced deficit in PPI. See FIG. 2.

In terms of startle magnitude, treatment of rats with PCP did not alter the actual magnitude of the startle responses. At the doses used, Clozapine and (2S)-2-(2-Bromo-3-oxo-spiro[3.5]non-1-en-1-ylamino)-3-[4-([2,7]naphthyridin-1-ylamino)phenyl]proprionic acid did not alter the magnitude of the startle response compared to the positive control group.

### DEFINITIONS

The term “therapeutically effective amount” as used in “a therapeutically effective amount of a compound of Formula 1 and/or Formula 2,” for example, refers to an amount of a compound of the present invention that is capable of measurably alleviating, partially or completely, one or more symptoms of schizophrenia in that subject.

As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

1. A method of treating schizophrenia or a symptom of schizophrenia or a positive symptom of schizophrenia in a psychotic disease, comprising administering a pharmaceutical composition to a mammal in need of such treatment, wherein the pharmaceutical composition comprises a therapeutically effective amount of a compound of Formula 1: 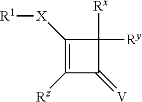
wherein
R¹ is a group Ar¹L²Ar²Alk-;
Ar¹ is a naphthyridynyl group optionally substituted with one or more -L³(Alk²)ₜL⁴(R⁴)ᵤ atoms or groups;
L³ and L⁴ are each, independently, a covalent bond, —O—, —S—, —C(O)—, —C(O)O—, —OC(O)—, —C(S)—, —S(O)—, —S(O)₂—, —N(R⁸)—, —CON(R⁸)—, —OC(O)N(R⁸)—, —CSN(R⁸)—, —N(R⁸)CO—, —N(R⁸)C(O)O—, —N(R⁸)CS—, —S(O)₂N(R⁸)—, —N(R⁸)S(O)₂—, —N(R⁸)O—, —ON(R⁸)—, —N(R⁸)N(R⁸)—, —N(R⁸)CON(R⁸)—, —N(R⁸)CSN(R⁸)—, or —N(R⁸)SO₂N(R⁸)—;
R⁸ is a hydrogen atom or a straight or branched C₁₋₆alkyl group optionally substituted with one, two, or three substituents selected from halogen, hydroxy and C₁₋₆alkoxy;
t is zero or the integer 1;
u is an integer 1, 2 or 3;
Alk² is an aliphatic or heteroaliphatic chain optionally substituted with one or more substituents selected from halogen, —OH, —CO₂H, —CO₂R⁹, —CONHR⁹, —CON(R⁹)₂, —COR⁹, C₁₋₆alkoxy, thiol, —S(O)R⁹, —S(O)₂R⁹, C₁₋₆ alkylthio, amino, —NHR⁹ and —N(R⁹)₂;
R⁹ is a straight or branched C₁₋₆alkyl group optionally substituted with one, two, or three substituents selected from halogen, hydroxy and C₁₋₆alkoxy;
R⁴ is a hydrogen atom; a halogen atom; C₁₋₆alkyl optionally substituted with one, two, or three substituents selected from halogen, hydroxy and C₁₋₆alkoxy; C₃₋₈cycloalkyl optionally substituted with one, two, or three substituents selected from halogen, hydroxy and C₁₋₆alkoxy; —OR⁵; —SR⁵; —NR⁵R⁶; —NO₂; —CN; —CO₂R⁵; —SO₃H; —SOR⁵; —SO₂R⁵; —SO₃R⁵; —OCO₂R⁵; —CONR⁵R⁶; —OCONR⁵R⁶; —CSNR⁵R⁶; —COR⁵; —OCOR⁵; —N(R⁵)COR⁶; —N(R⁵)CSR⁶; —SO₂N(R⁵)(R⁶); —N(R⁵)SO₂R⁶; N(R⁵)CON(R⁶)(R⁷); —N(R⁵)CSN(R⁶)(R⁷); or —N(R⁵)SO₂N(R⁶)(R⁷), provided that when t is zero and each of L³ and L⁴ is a covalent bond, then u is the integer 1 and R⁴ is other than a hydrogen atom;
R⁵, R⁶, R⁷, and R¹¹ are each, independently, a hydrogen atom or a C₁₋₆alkyl or C₃₋₈cycloalkyl group, wherein each of said alkyl and cycloalkyl groups is optionally substituted with one, two, or three substituents selected from halogen, hydroxy and C₁₋₆alkoxy;
L² is an N(R⁸) group;
Ar² is an arylene or heteroarylene group optionally substituted with one or more -L³(Alk²)ₜL⁴(R⁴)ᵤ atoms or groups;
Alk is a chain —CH₂—CH(R)—, —CH═C(R)—, or 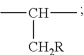
R is a carboxylic acid (—CO₂H), a carboxylic acid ester (—CO₂Alk⁷), a carboxylic acid amide (—CONR⁵R⁶), a tetrazole, phosphonic acid, phosphinic acid, sulphonic acid, sulphinic acid, boronic acid, or an acylsulphonamide group;
Alk⁷ is a straight or branched C₁₋₈alkyl group, C₂₋₈alkenyl group, C₂₋₈alkynyl group, C₃₋₈cycloalkyl group, C₃₋₈heterocycloalkyl group, C₃₋₈cycloalkylC₁₋₈alkyl group, C₃₋₈heterocycloalkylC₁₋₈alkyl group, C₁₋₆alkyloxyC₁₋₆alkyl group, hydroxyC₁₋₆alkyl group, C₁₋₆alkylthioC₁₋₆alkyl group, C₁₋₆alkylsulfinylC₁₋₆alkyl group, C₁₋₆alkylsulfonylC₁₋₆alkyl group, C₃₋₈cycloalkyloxyC₁₋₆alkyl group, C₃₋₈cycloalkylthioC₁₋₆alkyl group, C₃₋₈cycloalkylsulfinylC₁₋₆alkyl group, C₃₋₈cycloalkylsulfonylC₁₋₆alkyl group, C₁₋₆alkyloxycarbonylC₁₋₆alkyl group, C₁₋₆alkyloxycarbonylC₁₋₆alkenyl group, C₁₋₆alkyloxycarbonyloxyC₁₋₆alkyl group, C₁₋₆alkyloxycarbonyloxyC₁₋₆alkenyl group, C₃₋₈cycloalkyloxycarbonyloxyC₁₋₆alkyl group, N-di-C₁₋₈alkylaminoC₁₋₈alkyl group, N—C₆₋₁₂aryl-N—C₁₋₆alkylaminoC₁₋₆alkyl group, N-di-C₁₋₈alkyl-carbamoylC₁₋₈alkyl group, C₆₋₁₂arylC₁₋₆alkyl group, heteroC₆₋₁₀arylC₁₋₆alkyl group, C₆₋₁₂aryl group, a C₆₋₁₂aryloxyC₁₋₈alkyl group, a C₆₋₁₂arylthioC₁₋₈alkyl group, a C₆₋₁₂arylsulfinylC₁₋₈alkyl group, a C₆₋₁₂arylsulfonylC₁₋₈alkyl group, C₁₋₈alkanoyloxyC₁₋₈alkyl group, C₄₋₈imidoC₁₋₈alkyl group, a C₆₋₁₂aroyloxyC₁₋₈alkyl group, or a triglyceride, optionally substituted with one or more R^{13a} groups;
R^{13a} is a halogen atom, or an amino (—NH₂), NHR¹⁴, —N(R¹⁴)₂, nitro, cyano, amidino, hydroxyl (—OH), —OR¹⁴, formyl, carboxyl (—CO₂H), esterified carboxyl, thiol (—SH), —SR¹⁴, SC(═NH)NH₂, —COR¹⁴, CSR¹⁴, —SO₃H, —SOR¹⁴, —SO₂R¹⁴, —SO₃R¹⁴, —SO₂NH₂, —SO₂NHR¹⁴, —SO₂N(R¹⁴)₂, —CONH₂, —CSNH₂, —CONHR¹⁴, —CSNHR¹⁴, —CON(R¹⁴)₂, —CSN(R¹⁴)₂, —N(R¹¹)SO₂R¹⁴, —N(SO₂R¹⁴)₂, —NH(R¹¹)SO₂NH₂, —N(R¹¹)SO₂NHR¹⁴, —N(R¹¹)SO₂N(R¹⁴)₂, —N(R¹¹)COR¹⁴, —N(R¹¹)CONH₂, —N(R¹¹)CONHR¹⁴, —N(R¹¹)CON(R¹⁴)₂, N(R¹¹)CSNH₂, —N(R¹¹)CSNHR114, N(R¹¹)CSN(R¹⁴)₂, —N(R¹¹)CSR¹⁴, —N(R¹¹)C(O)OR¹⁴, —SO₂NHet¹, —CONHet¹, —CSNHet¹, —N(R¹¹)SO₂NHet¹, —N(R¹¹)CONHet¹, —N(R¹¹)CSNHet¹, —SO₂N(R¹¹)Het², -Het², —CON(R¹¹)Het², —CSN(R¹¹)Het², —N(R¹¹)CON(R¹¹)Het², —N(R¹¹)CSN Het², aryl or heteroaryl group;
R¹⁴ is an -Alk⁶(R^{13a})ₘ, aryl, or heteroaryl group;
Alk⁶ is a straight or branched C₁₋₆alkylene, C₂₋₆alkenylene or C₂₋₆alkynylene chain, optionally interrupted by one, two or three —O— or —S— atoms or —S(O)ₚ or —N(R¹⁵)— groups;
m is zero or the integer 1, 2 or 3;
p is an integer 1 or 2;
R¹⁵ is a hydrogen atom or C₁₋₆alkyl group;
Het¹ is a C₅₋₇cyclicamino group optionally containing one or more —O— or —S— atoms or —N(R¹¹)—, —C(O)—, —C(S)—, —S(O) or —S(O)₂ groups and optionally substituted with one or more substituents selected from halogen, —OH, —CO₂H, —CO₂R⁹, CONHR⁹, —CON(R⁹)₂, —COR⁹, C₁₋₆alkoxy, thiol, —S(O)R⁹, —S(O)₂R⁹, C₁₋₆alkylthio, amino, —NHR⁹ and —N(R⁹)₂;
Het² is a monocyclic C₅₋₇carbocyclic group optionally containing one or more —O— or —S— atoms or —N(R¹¹)—, —C(O)— or —C(S)— groups and optionally substituted with one or more substituents selected from halogen, —OH, —CO₂H, —CO₂R⁹, —CONHR⁹, —CON(R⁹)₂, —COR⁹, C₁₋₆alkoxy, thiol, —S(O)R⁹, —S(O)₂R⁹, C₁₋₆alkylthio, amino, —NHR⁹ and —N(R⁹)₂;
X is an —O— or —S— atom or an —N(R²)— group;
R² is a hydrogen atom or a C₁₋₆alkyl group;
V is an oxygen (O) or sulfur (S) atom;
R^{z} is an atom or group -L¹(Alk¹)ₙ(R³)ᵥ;
L¹ is a covalent bond, an —O—, —S—, or —Se— atom, or a —C(O)—, —C(O)O—, —OC(O)—, —C(S)—, —S(O)—, —S(O)₂—, —N(R⁸)—, —CON(R⁸)—, —OC(O)N(R⁸)—, —CSN(R⁸)—, —N(R⁸)CO—, —N(R⁸)C(O)O—, —N(R⁸)CS—, S(O)₂N(R⁸)—, —N(R⁸)S(O)₂—, —N(R⁸)O—, —ON(R⁸)—, —N(R⁸)N(R⁸)—, —N(R⁸)CON(R⁸)—, —N(R⁸)CSN(R⁸)—, or —N(R⁸)SO₂N(R⁸)— group;
Alk¹ is an aliphatic or heteroaliphatic chain optionally substituted with one or more substituents selected from halogen, —OH, —CO₂H, —CO₂R⁹, —CONHR⁹, —CON(R⁹)₂, —COR⁹, C₁₋₆alkoxy, thiol, —S(O)R⁹, —S(O)₂R⁹, C₁₋₆alkylthio amino, —NHR⁹ and —N(R⁹)₂;
R³ is a hydrogen or halogen atom or a group selected from —OR^{3a}, —SR^{3a}, —CN and a C₃₋₁₀cycloalkyl; C₃₋₁₀cycloalkenyl; C₃₋₁₀heterocycloalkyl or C₃₋₁₀heterocycloalkenyl containing 1, 2, 3 or 4 heteroatoms or heteroatom-containing groups L⁵, where L⁵ is defined as for L³; aromatic or heteroaromatic group optionally substituted with one or more substituents selected from halogen, C₁₋₆alkyl, haloC₁₋₆alkyl, —C(OH)(CF₃)₂, C₁₋₆alkoxy, haloC₁₋₆alkoxy, thiol, C₁₋₆alkylthio, -(Alk⁴)gR¹⁰, —CN, —CO₂R¹¹, —NO₂, —CON(R¹¹)₂, —CSN(R¹¹)₂, —COR¹¹, —CSN(R¹¹)₂, —N(R¹¹)COR¹¹, —N(R¹¹)CSR¹¹, —SO₂N(R¹¹)₂, N(R¹¹)SO₂R¹¹, —N(R¹¹)CON(R¹¹)₂, —N(R¹¹)CSN(R¹¹), N(R¹¹)SO₂N(R¹¹)₂, and phenyl optionally substituted with one, two or three R¹³ groups;
R¹³ is —R^{13a} or -Alk⁶(R^{13a})ₘ;
Alk⁴ is a straight or branched C₁₋₃alkylene chain;
g is zero or an integer 1;
R¹⁰ is —OH, —SH, or —N(R¹¹)₂;
R^{3a} is a hydrogen atom or a straight or branched C₁₋₆alkyl group or C₃₋₈cycloalkyl group, wherein each of said alkyl and cycloalkyl groups is optionally substituted with one, two, or three substituents selected from halogen, hydroxy, and C₁₋₆alkoxy;
n is zero or the integer 1;
v is the integer 1, 2 or 3;
provided that when n is zero and L¹ is a covalent bond, v is the integer 1;
R^{X} and R^{Y}, together with the carbon atom to which they are attached, are joined together to form a spiro linked cyclopentyl, cyclohexyl, cycloheptyl, or tetrahydropyranyl group optionally substituted with one or more substituents selected from halogen, C₁₋₆alkyl, haloC₁₋₆alkyl, —C(OH)(CF₃)₂, C₁₋₆alkoxy, haloC₁₋₆alkoxy, thiol, C₁₋₆alkylthio, -(Alk⁴)gR¹⁰, —CN, —CO₂R¹¹, —NO₂, —CON(R¹¹)₂, —CSN(R¹¹)₂, —COR¹¹, —CSN(R¹¹)₂, —N(R¹¹)COR¹¹, —N(R¹¹)CSR¹¹, —SO₂N(R¹¹)₂, —N(R¹¹)SO₂R¹¹, —N(R¹¹)CON(R¹¹)₂, —N(R¹¹)CSN(R¹¹), N(R¹¹)SO₂N(R¹¹)₂, and phenyl optionally substituted with one, two or three R¹³ groups;
or a salt, solvate, hydrate or N-oxide thereof.

2. The method of treating schizophrenia or a symptom of schizophrenia or a positive symptom of schizophrenia in a psychotic disease according to claim 1, in which Alk is a —CH₂CH(R)— or —CH(CH₂R)— chain.

3. The method of treating schizophrenia or a symptom of schizophrenia or a positive symptom of schizophrenia in a psychotic disease according to claim 1, in which R is a carboxylic acid (—CO₂H) group.

4. The method of treating schizophrenia or a symptom of schizophrenia or a positive symptom of schizophrenia in a psychotic disease according to claim 1, in which R is a carboxylic acid ester of formula —CO₂Alk⁷.

5. The method of treating schizophrenia or a symptom of schizophrenia or a positive symptom of schizophrenia in a psychotic disease according to claim 1, in which X is an —N(R²)— group.

6. The method of treating schizophrenia or a symptom of schizophrenia or a positive symptom of schizophrenia in a psychotic disease according to claim 5, in which R² is a hydrogen atom.

7. The method of treating schizophrenia or a symptom of schizophrenia or a positive symptom of schizophrenia in a psychotic disease according to claim 1, in which Ar₂ is an optionally substituted phenylene group or an optionally substituted pyridinediyl group of formula: 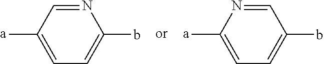
where a and b signify the points of attachment of L² and Alk respectively.

8. The method of treating schizophrenia or a symptom of schizophrenia or a positive symptom of schizophrenia in a psychotic disease according to claim 1, in which R^{z} is a halogen atom.

9. The method of treating schizophrenia or a symptom of schizophrenia or a positive symptom of schizophrenia in a psychotic disease according to claim 1, in which R^{z} is an optionally substituted C₁₋₈ alkyl group.

10. The method of treating schizophrenia or a symptom of schizophrenia or a positive symptom of schizophrenia in a psychotic disease according to claim 1, in which R^{z} is a group -L¹(Alk¹)ₙR³ in which L¹ is an —O—, —S— or —Se— atom or —S(O)— or —N(R⁸)— group.

11. The method of treating schizophrenia or a symptom of schizophrenia or a positive symptom of schizophrenia in a psychotic disease according to claim 10, in which n is zero.

12. The method of treating schizophrenia or a symptom of schizophrenia or a positive symptom of schizophrenia in a psychotic disease according to claim 10, in which n is the integer 1 and Alk¹ is an optionally substituted C₁₋₆alkylene chain.

13. The method of treating schizophrenia or a symptom of schizophrenia or a positive symptom of schizophrenia in a psychotic disease according to claim 10, in which R³ is a hydrogen atom or an optionally substituted C₃₋₁₀cycloalkyl; C₃₋₁₀cycloalkenyl; C₃₋₁₀heterocycloalkyl or C₃₋₁₀heterocycloalkenyl containing 1, 2, 3 or 4 heteroatoms or heteroatom-containing groups L⁵, where L⁵ is defined as for L³.

14. The method of treating schizophrenia or a symptom of schizophrenia or a positive symptom of schizophrenia in a psychotic disease according to claim 1, in which R^{z} is a group -L¹(Alk¹)ₙR³ in which L¹ is a covalent bond.

15. The method of treating schizophrenia or a symptom of schizophrenia or a positive symptom of schizophrenia in a psychotic disease according to claim 14, in which n is zero.

16. The method of treating schizophrenia or a symptom of schizophrenia or a positive symptom of schizophrenia in a psychotic disease according to claim 14, in which n is the integer 1 and Alk¹ is an optionally substituted C₁₋₆alkylene chain.

17. The method of treating schizophrenia or a symptom of schizophrenia or a positive symptom of schizophrenia in a psychotic disease according to claim 14, in which R³ is a hydrogen atom or an optionally substituted C₃₋₁₀cycloalkyl; C₃₋₁₀cycloalkenyl; C₃₋₁₀heterocycloalkyl or C₃₋₁₀heterocycloalkenyl containing 1, 2, 3 or 4 heteroatoms or heteroatom-containing groups L⁵, where L⁵ is defined as for L³.

18. The method of treating schizophrenia or a symptom of schizophrenia or a positive symptom of schizophrenia in a psychotic disease according to claim 1, in which R^{x} and R^{y} are joined to form an optionally substituted cyclohexyl group.

19. The method of treating schizophrenia or a symptom of schizophrenia or a positive symptom of schizophrenia in a psychotic disease according to claim 1, in which the compound of Formula 1 is selected from the group consisting of:
(2 S)-2-[(2-Isopropylsulfanyl-3-oxo-spiro[3.5]non-1-en-1-yl)amino]-3-[4-([2,7]naphthyridin-1-ylamino)phenyl]propanoic acid;
(2 S)-2-[(2-Isopropylsulfanyl-3-oxo-7-oxa-spiro[3.5]non-1-en-1-yl)amino]-3-[4-([2,7]naphthyridin-1-ylamino)phenyl]propanoic acid;
(2 S)-2-(2-Bromo-3-oxo-spiro[3.5]non-1-en-1-ylamino)-3-[4-(3-methyl-[2,7]naphthyridin-1-ylamino)phenyl]propanoic acid;
(2 S)-2-(2-Bromo-3-oxo-spiro[3.5]non-1-en-1-ylamino)-3-[4-([2,7]naphthyridin-1-ylamino)phenyl]propanoic acid;
and salts, solvates, hydrates, N-oxides or carboxylic acid esters thereof.

20. The method of treating schizophrenia or a symptom of schizophrenia or a positive symptom of schizophrenia in a psychotic disease according to claim 1, in which Ar¹ is an optionally substituted 2,7-naphthyridinyl group.

21. The method of treating schizophrenia or a symptom of schizophrenia or a positive symptom of schizophrenia in a psychotic disease according to claim 1, wherein the mammal is a human.

22. A method of treating schizophrenia or a symptom of schizophrenia or a positive symptom of schizophrenia in a psychotic disease, comprising administering a pharmaceutical composition to a mammal in need of such treatment, wherein the pharmaceutical composition comprises a therapeutically effective amount of a compound of Formula 2: 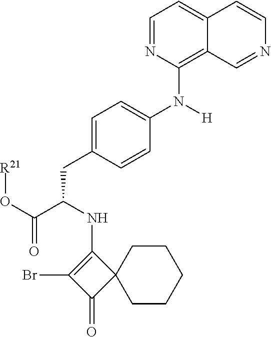
wherein R²¹ is H; a straight or branched C₁₋₆alkyl group optionally substituted with one, two or three substituents selected from halogen, hydroxy and C₁₋₆alkoxy; —CH₂CH₂OCH₃; —CH₂CH₂OCH₂CH₂OH; —CH₂CH₂OCH₂CH₂OCH₃; 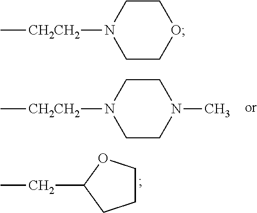
or the salts, solvates or N-oxides thereof.

23. The method of treating schizophrenia or a symptom of schizophrenia or a positive symptom of schizophrenia in a psychotic disease according to embodiment 22, wherein R²¹ is H, straight C₁₋₆alkyl, —CH(CH₃)₂, —(CH₂)₂CH₃, —CH₂C(CH₃)₃—CH₂CH₂OH, —CH₂CH₂OCH₃, —CH₂CH₂OCH₂CH₂OH, —CH₂CH₂OCH₂CH₂OCH₃,

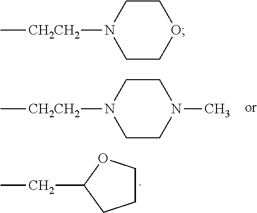

24. The method of treating schizophrenia or a symptom of schizophrenia or a positive symptom of schizophrenia in a psychotic disease according to claim 22, wherein the mammal is a human.

25. The method of treating schizophrenia or a symptom of schizophrenia or a positive symptom of schizophrenia in a psychotic disease according to claim 23, wherein the mammal is a human.

26. The method of treating a positive symptom of schizophrenia in a psychotic disease according to claim 1, wherein the symptom is bipolar disorder, delusional disorder, psychotic depression, Tourette syndrome, autism spectrum disorder, OCD, dementia or Alzheimer's disease.

27. The method of treating a positive symptom of schizophrenia in a psychotic disease according to claim 22, wherein the symptom is bipolar disorder, delusional disorder, psychotic depression, Tourette syndrome, autism spectrum disorder, OCD, dementia or Alzheimer's disease.
